(19) United States
(12) Patent Application Publication
(10) Pub. No.: US 2005/0282838 A1
(43) Pub. Date: Dec. 22, 2005

(54) Compounds, compositions, and methods

(76) Inventors: Shyamlal Ramchandani, San Francisco, CA (US); Reginald Norman De La Rosa, Concord, CA (US); Cynthia L. Adams, San Carlos, CA (US); Gustave Bergnes, Pacifica, CA (US); David J. Morgans JR., Los Altos, CA (US); Jay K. Trautman, Los Altos, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/966,348

(22) Filed: Oct. 15, 2004

Related U.S. Application Data
(60) Provisional application No. 60/512,494, filed on Oct. 16, 2003.

(51) Int. Cl. A61K 31/513

(52) U.S. Cl. 514/269; 544/315

(57) ABSTRACT

Compounds useful for treating cellular proliferative diseases are disclosed.

This application claims the benefit of co-pending provisional U.S. Applications No. 60/512,494, filed Oct. 16, 2003, which is incorporated herein by reference.

Compounds which are useful in the treatment of cellular proliferative diseases, for example cancer, hyperplasias, restenosis, cardiac hypertrophy, immune disorders, fungal disorders, and inflammation are described as well as pharmaceutical compositions and methods for their use.

Improvements in the specificity of agents used to treat cancer is of considerable interest because of the therapeutic benefits which would be realized if the side effects associated with the administration of these agents could be reduced. Traditionally, dramatic improvements in the treatment of cancer are associated with identification of therapeutic agents acting through novel mechanisms. Examples of this include not only the taxanes, but also the camptothecin class of topoisomerase I inhibitors.

Accordingly, it is an object of the present invention to provide compounds, compositions and methods useful in the treatment of cellular proliferative diseases.

In accordance with the objects outlined above, compounds that can be used to treat cellular proliferative diseases are provided, as well as methods for treating cellular proliferative diseases. The methods employ one or more compounds represented by Formula I: 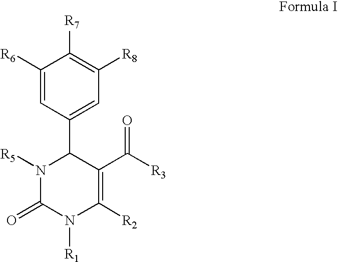
wherein:
R₁ and R₅ are each independently hydrogen, optionally substituted lower alkyl, optionally substituted aryl or optionally substituted heteroaryl;
R₂ is hydrogen, optionally substituted lower alkyl, optionally substituted aryl or optionally substituted heteroaryl;
R₃ is optionally substituted alkoxy, optionally substituted alkyl, or NR₉R₁₀ wherein R₉ and R₁₀ are independently selected from hydrogen, optionally substituted lower alkyl, optionally substituted aryl or optionally substituted heteroaryl;
R₆ is hydrogen, cyano, nitro, halo, optionally substituted alkyl, or optionally substituted alkoxy;
R₇ is halo, optionally substituted alkyl, cyano, nitro, hydroxy, optionally substituted alkoxy, or optionally substituted amino; and
R₈ is halo, optionally substituted alkyl, or optionally substituted alkoxy;
or R₆ and R₇, together with the carbons to which they are attached, form an optionally substituted 3- to 7-membered ring which optionally includes one or more heteroatoms selected from N, O, and S in the ring; and R₈ is halo, optionally substituted alkyl, or optionally substituted alkoxy;
or R₇ and R₈, together with the carbons to which they are attached, form an optionally substituted 3- to 7-membered ring which optionally includes one or more heteroatoms selected from N, O, and S in the ring; and R₆ is hydrogen, cyano, nitro, halo, optionally substituted alkyl, or optionally substituted alkoxy;
provided that only one of R₆, R₇, and R₈ is optionally substituted alkoxy; and
provided that when R₁ and R₅ are hydrogen, R₂ is methyl, R₃ is methoxy or ethoxy, R₇ is hydroxy, and R₈ is methoxy, then R₆ is not hydrogen or halo.

In one aspect, methods for treating cellular proliferative diseases by the administration of a therapeutically effective amount of a compound of Formula I are provided. Such diseases and disorders include cancer, hyperplasia, restenosis, cardiac hypertrophy, immune disorders, fungal disorders and inflammation.

In another aspect, pharmaceutical compositions comprising: a therapeutically effective amount of a compound of Formula I; and one or more pharmaceutical excipients are provided. In another aspect, the composition further comprises a chemotherapeutic agent other than a compound of the present invention.

As used in the present specification, the following words and phrases are generally intended to have the meanings as set forth below, except to the extent that the context in which they are used indicates otherwise. The following abbreviations and terms have the indicated meanings throughout:
Ac=acetyl
Boc=t-butyloxy carbonyl
Bu=butyl
c-=cyclo
DCM=dichloromethane=methylene chloride=CH₂Cl₂
DIEA=N,N-diisopropylethylamine
DMF=N,N-dimethylformamide
DMSO=dimethyl sulfoxide
Et=ethyl
GC=gas chromatography
HOAc=acetic acid
Me=methyl
mesyl=methanesulfonyl
Ph=phenyl
PhOH=phenol
Py=pyridine
rt=room temperature
sat'd=saturated
s-=secondary
t-=tertiary
TES=triethylsilyl
TFA=trifluoroacetic acid
THF=tetrahydrofuran
TMS=trimethylsilyl
tosyl=p-toluenesulfonyl
Tf=triflate

The term “acyl” refers to groups of from 1 to 8 carbon atoms of a straight, branched, or cyclic configuration or a combination thereof, attached to the parent structure through a carbonyl functionality. Such groups may be saturated or unsaturated, and aliphatic or aromatic. One or more carbons in the acyl residue may be replaced by nitrogen, oxygen or sulfur as long as the point of attachment to the parent remains at the carbonyl. Examples include acetyl, benzoyl, propionyl, isobutyryl, t-butoxycarbonyl, benzyloxycarbonyl and the like.

The term “alkoxy” or “alkoxyl” refers to an alkyl group, such as including from 1 to 8 carbon atoms, of a straight, branched, or cyclic configuration, or a combination thereof, attached to the parent structure through an oxygen (i.e., the group alkyl-O—). Examples include methoxy-, ethoxy-, propoxy-, isopropoxy-, cyclopropyloxy-, cyclohexyloxy- and the like. Lower alkoxy refers to alkoxy groups containing one to four carbons.

The term “alkoxycarbonyl” refers to the group (alkyl)-O—C(O)— wherein the group is attached to the parent structure through the carbonyl functionality.

The term “alkyl” refers to linear, branched, and cyclic aliphatic hydrocarbon structures and combinations thereof, which structures may be saturated or unsaturated. In some embodiments, alkyl groups are those of C₂₀ or below. In some embodiments, alkyl groups are those of C₁₃ or below. Alkyl includes alkanyl, alkenyl and alkynyl residues; such as vinyl, allyl, isoprenyl and the like. When an alkyl residue having a specific number of carbons is named, all geometric isomers having that number of carbons are encompassed; thus, for example, “butyl” refers to n-butyl, sec-butyl, isobutyl and t-butyl; “propyl” includes n-propyl, isopropyl, and c-propyl.

Alkylene-, alkenylene-, and alkynylene- are subsets of alkyl, including the same residues as alkyl, but having two points of attachment within a chemical structure. Examples of alkylene include ethylene (—CH₂CH₂—), propylene (—CH₂CH₂CH₂—), dimethylpropylene (—CH₂C(CH₃)₂CH₂—) and cyclohexylpropylene (—CH₂CH₂CH(C₆H₁₃)—). Likewise, examples of alkenylene include ethenylene (—CH═CH—), propenylene (—CH═CH—CH₂—), and cyclohexylpropenylene (—CH═CHCH(C₆H₁₃)—). Examples of alkynylene include ethynylene (—C≡C—) and propynylene (—CH≡CH—CH₂—).

The term “amido” refers to the groups —NR^{c}C(O)R^{b}, —NR^{c}C(O)OR^{a}, and —NR^{c}C(O)NR^{b}R^{c}, where
R^{a} is chosen from optionally substituted C₁-C₆ alkyl, optionally substituted aryl, and optionally substituted heteroaryl;
R^{b} is chosen from H, optionally substituted C₁-C₆ alkyl, optionally substituted aryl, and optionally substituted heteroaryl; and
R^{c} is chosen from hydrogen and optionally substituted C₁-C₄ alkyl; and
where each substituted group is independently substituted with one or more substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halogen, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for heteroaryl), —CO²H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

The term “amino” refers to the group —NH₂.

The term “aminocarbonyl” refers to the group —CONR^{b}R^{c}, where
R^{b} is chosen from H, optionally substituted C₁-C₆ alkyl, optionally substituted aryl, and optionally substituted heteroaryl; and
R^{c} is chosen from hydrogen and optionally substituted C₁-C₄ alkyl; and
where each substituted group is independently substituted with one or more substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halogen, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for heteroaryl), —CO²H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

The term “aralkoxy” refers to the group —O-aralkyl. Similarly, heteroaralkoxy- refers to the group —O-heteroaralkyl; aryloxy- refers to the group —O-aryl; acyloxy- refers to the group —O-acyl; heteroaryloxy- refers to the group —O-heteroaryl; and heterocyclyloxy- refers to the group —O—heterocyclyl (i.e., thee aralkyl, heteroaralkyl, aryl, acyl, heterocycloalkyl, or heteroaryl is attached to the parent structure through an oxygen).

The term “aralkyl” refers to a residue in which an aryl moiety is attached to the parent structure via an alkyl residue. Examples include benzyl, phenethyl, phenylvinyl, phenylallyl and the like.

The term “aryl” refers to a 6-membered aromatic ring; a bicyclic 9 or 10-membered aromatic ring system in which at least one of the rings in the ring system is aromatic; and a tricyclic 12- to 14-membered aromatic ring system in which at least one of the rings in the ring system is aromatic. The aromatic 6- to 14-membered carbocyclic rings include, e.g., phenyl, naphthyl, indanyl, tetralinyl, and fluorenyl.

The term “cycloalkenyl” refers to unsaturated cyclic hydrocarbon groups of from 3 to 13 carbon atoms and is a subset of alkyl. Examples of cycloalkenyl groups include c-hexenyl-, c-pentenyl and the like.

The term “cycloalkyl” refers to cyclic aliphatic hydrocarbon groups of from 3 to 13 carbon atoms and is a subset of alkyl. Examples of cycloalkyl groups include c-propyl, c-butyl, c-pentyl, norbornyl, adamantyl and the like.

The term “cycloalkyl-alkyl-” refers to cycloalkyl attached to the parent structure through a non-cyclic alkyl and is another subset of alkyl. Examples of cycloalkyl-alkyl- include cyclohexylmethyl, cyclopropylmethyl, cyclohexylpropyl, and the like.

The term “halogen” or “halo” refers to fluorine (or fluoro), chlorine (or chloro), bromine (or bromo) or iodine (or iodo). Dihaloaryl, dihaloalkyl, trihaloaryl etc. refer to aryl and alkyl substituted with the designated plurality of halogens (here, 2, 2 and 3, respectively), but not necessarily a plurality of the same halogen; thus 4-chloro-3-fluorophenyl is within the scope of dihaloaryl.

The term “heteroaralkyl” refers to a residue in which a heteroaryl moiety is attached to the parent structure via an alkyl residue. Examples include furanylmethyl, pyridinylmethyl, pyrimidinylethyl and the like.

The term “heteroaryl” refers to
a 5- or 6-membered aromatic heterocyclic ring containing 1-4 heteroatoms selected from O, N, or S;
a bicyclic 9- or 10-membered ring system in which at least one of the rings in the ring system is aromatic and contains 1-4 heteroatoms selected from O, N, or S; and
a tricyclic 12- to 14-membered ring system in which at least one of the rings in the ring system is aromatic and contains 1-4 heteroatoms selected from O, N, or S. The 5- to 10-membered aromatic heterocyclic rings, i.e., heteroaryl groups, include, e.g., imidazolyl, pyridinyl, indolyl, thienyl, benzopyranonyl, thiazolyl, furanyl, benzimidazolyl, quinolinyl, isoquinolinyl, quinoxalinyl, pyrimidinyl, pyrazinyl, tetrazolyl and pyrazolyl.

The term “heterocycloalkyl” or “heterocyclyl” refers to a cycloalkyl residue in which one to four of the carbons is replaced by a heteroatom such as oxygen, nitrogen or sulfur. Examples include pyrrolidine, tetrahydrofuran, tetrahydro-thiophene, thiazolidine, piperidine, tetrahydro-pyran, tetrahydro-thiopyran, piperazine, morpholine, thiomorpholine and dioxane. Heterocyclyl also includes ring systems including unsaturated bonds, provided the number and placement of unsaturation does not render the group aromatic. Examples include imidazoline, oxazoline, tetrahydroisoquinoline, benzodioxan, benzodioxole and 3,5-dihydrobenzoxazinyl.

The term “leaving group” refers to any group (or atom) that will, under the reaction conditions, cleave from the starting material, thus promoting reaction at a specified site. Suitable examples of such groups unless otherwise specified are halogen atoms, mesyloxy, p-nitrobenzensulphonyloxy and tosyloxy groups.

The term “lower alkyl” refers to alkyl groups of from 1 to 5 carbon atoms, such as from 1 to 4 carbon atoms. Examples of lower alkyl groups include methyl, ethyl, propyl, isopropyl, butyl, s-and t-butyl and the like.

The term “optional” or “optionally” means that the subsequently described event or circumstance may or may not occur, and that the description includes instances where said event or circumstances occurs and instances in which it does not. For example, “optionally substituted alkyl” includes “alkyl” and “substituted alkyl” as defined herein. It will be understood by those skilled in the art with respect to any group containing one or more substituents that such groups are not intended to introduce any substitution or substitution patterns that are sterically impractical and/or synthetically non-feasible and/or inherently unstable.

The term “pharmaceutically acceptable salts” refers to those salts that retain the biological effectiveness of the free compound and that are not biologically or otherwise undesirable, formed with a suitable acid or base, and includes pharmaceutically acceptable acid addition salts and base addition salts.

The term “pharmaceutically acceptable acid addition salts” refers to pharmaceutically acceptable salts derived from inorganic acids such as hydrochloric acid, hydrobromic acid, sulfuric acid, nitric acid, phosphoric acid and the like, and those derived from organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

The term “pharmaceutically acceptable base addition salts” refer to pharmaceutically acceptable salts derived from inorganic bases such as sodium, potassium, lithium, ammonium, calcium, magnesium, iron, zinc, copper, manganese, aluminum salts and the like. In some embodiments, the pharmaceutically acceptable base addition salt is chosen from ammonium, potassium, sodium, calcium, and magnesium salts. Base addition salts also include those derived from pharmaceutically acceptable organic non-toxic bases, including salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The term “prodrug” refers to a compound that is a drug precursor that, following administration and absorption, release the drug in vivo by a metabolic process. See, e.g., Ettmayer et al. (2004) J. Med. Chem. 47:2393-2404.

The term “protecting group” has the meaning conventionally associated with it in organic synthesis, i.e. a group that selectively blocks one or more reactive sites in a multifunctional compound such that a chemical reaction can be carried out selectively on another unprotected reactive site and such that the group can readily be removed after the selective reaction is complete. A variety of protecting groups are disclosed, for example, in T. H. Greene and P. G. M. Wuts, Protective Groups in Organic Synthesis, Third Edition, John Wiley & Sons, New York (1999). For example, a hydroxy protected form is where at least one of the hydroxyl groups present in a compound is protected with a hydroxy protecting group. Likewise, amines and other reactive groups may similarly be protected.

The term “solvate” refers to the compound formed by the interaction of a solvent and a compound. Suitable solvates are pharmaceutically acceptable solvates, such as hydrates, including monohydrates and hemi-hydrates.

The term “substituted-” alkyl, aryl, and heteroaryl refer respectively to alkyl, aryl, and heteroaryl wherein one or more (up to about 5, such as up to about 3) hydrogen atoms are replaced by a substituent independently selected from the group:
—R^{a}, —OR^{b}, —O(C₁-C₂ alkyl)O— (e.g., methylenedioxy-), —SR^{b}, guanidine, guanidine wherein one or more of the guanidine hydrogens are replaced with a lower alkyl group, —NR_{b}R_{c}, halogen, cyano, nitro, —COR^{b}, —CO²R^{b}, —CONR^{b}R_{c}, —OCOR^{b}, —OCO²R^{a}, —OCONR^{b}R_{c}, —NR^{c}COR^{b}, —NR^{c}CO²R, —NR^{c}CONR^{b}R_{c}, —CO²R^{b}, —CONR^{b}R_{c}, —NR^{c}COR^{b}, —SOR^{a}—SO₂R^{a}—SO₂NR_{b}R_{c}, and —NR^{c}SO₂R^{a},
where R^{a} is chosen from optionally substituted C₁-C₆ alkyl, optionally substituted aryl, and optionally substituted heteroaryl;
R^{b} is chosen from H, optionally substituted C₁-C₆ alkyl, optionally substituted aryl, and optionally substituted heteroaryl; and
R^{c} is chosen from hydrogen and optionally substituted C₁-C₄ alkyl;
where each substituted group is independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halogen, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for heteroaryl), —CO²H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

The term “substituted acyl” refers to the groups (substituted alkyl)-C(O)—; (substituted aryl)-C(O)—; (substituted heteroaryl)-C(O)—; and (substituted heterocycloalkyl)-C(O)—, wherein the group is attached to the parent structure through the carbonyl functionality. One or more carbons in the substituted acyl residue may be replaced by nitrogen, oxygen or sulfur as long as the point of attachment to the parent remains at the carbonyl.

The term “substituted alkoxy” refers to alkoxy wherein the alkyl constituent is substituted (i.e., —O-(substituted alkyl)). In some embodiments, a substituted alkoxy group is “polyalkoxy” or —O-(optionally substituted alkylene)-(optionally substituted alkoxy), and includes groups such as —OCH₂CH₂OCH₃, and residues of glycol ethers such as polyethyleneglycol, and —O(CH₂CH₂O)ₓCH₃, where x is an integer of about 2-20, such as about 2-10, and for example, about 2-5. Another substituted alkoxy group is hydroxyalkoxy or —OCH₂(CH₂)_{y}OH, where y is an integer of about 1-10, such as about 1-4.

The term “substituted alkoxycarbonyl” reers to the group (substituted alkyl)-O—C(O)— wherein the group is attached to the parent structure through the carbonyl functionality.

The term “substituted amino” refers to the group —NHR or —NRR where each R is independently selected from the group: optionally substituted alkyl, optionally substituted alkoxy, optionally substituted amino carbonyl, optionally substituted aryl, optionally substituted heteroaryl, optionally substituted heterocycloalkyl, acyl, alkoxycarbonyl, sulfinyl and sulfonyl, e.g., diethylamino, methylsulfonylamino, furanyl-oxy-sulfonamino.

The term “sulfanyl” refers to the groups: —S-(optionally substituted alkyl), —S-(optionally substituted aryl), —S-(optionally substituted heteroaryl), and —S-(optionally substituted heterocycloalkyl).

The term “sulfinyl” refers to the groups: —S(O)—H, —S(O)-(optionally substituted alkyl), —S(O)-optionally substituted aryl), —S(O)-optionally substituted heteroaryl), —S(O)-(optionally substituted heterocycloalkyl); and —S(O)-(optionally substituted amino).

The term “sulfonamido” refers to the group —NR^{c}S(O₂)R^{a} where
R^{a} is chosen from optionally substituted C₁-C₆ alkyl, optionally substituted aryl, and optionally substituted heteroaryl; and
R^{c} is chosen from hydrogen and optionally substituted C₁-C₄ alkyl.

The term “sulfonyl” refers to the groups: —S(O₂)—H, —S(O₂)-(optionally substituted alkyl), —S(O₂)-optionally substituted aryl), —S(O₂)-optionally substituted heteroaryl), —S(O₂)-(optionally substituted heterocycloalkyl), —S(O₂)-(optionally substituted alkoxy), —S(O₂)-optionally substituted aryloxy), —S(O₂)-optionally substituted heteroaryloxy), —S(O₂)-(optionally substituted heterocyclyloxy); and —S(O₂)-(optionally substituted amino).

Many of the compounds described herein contain one or more asymmetric centers and may thus give rise to enantiomers, diastereomers, and other stereoisomeric forms that may be defined, in terms of absolute stereochemistry, as (R)- or (S)-. The present invention is meant to include all such possible isomers, including racemic mixtures, optically pure forms and intermediate mixtures. Optically active (R)- and (S)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms and rotational isomers are also intended to be included.

When desired, the (R)- and (S)-isomers may be resolved by methods known to those skilled in the art, for example by formation of diastereoisomeric salts or complexes which may be separated, for example, by crystallization; via formation of diastereoisomeric derivatives which may be separated, for example, by crystallization, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where the desired enantiomer is converted into another chemical entity by one of the separation procedures described above, a further step may be required to liberate the desired enantiomeric form. Alternatively, specific enantiomer may be synthesized by asymmetric synthesis using optically active reagents, substrates, catalysts or solvents, or by converting one enantiomer to the other by asymmetric transformation.

Compounds

The present invention is directed to a class of novel compounds that cause mitotic arrest and cell death for the treatment of disorders associated with cell proliferation. The compounds, compositions and methods described herein can differ in their selectivity and are used to treat diseases of cellular proliferation, including, but not limited to cancer, hyperplasias, restenosis, cardiac hypertrophy, immune disorders, fungal disorders and inflammation.

Accordingly, the present invention relates to compositions comprising and methods employing compounds represented by Formula I: 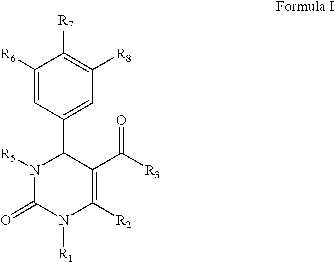
wherein
R₁ and R₅ are each independently hydrogen, optionally substituted lower alkyl, optionally substituted aryl or optionally substituted heteroaryl;
R₂ is hydrogen, optionally substituted lower alkyl, optionally substituted aryl or optionally substituted heteroaryl;
R₃ is optionally substituted alkoxy, optionally substituted alkyl, or NR₉R₁₀ wherein R₉ and R₁₀ are independently selected from hydrogen, optionally substituted lower alkyl, optionally substituted aryl or optionally substituted heteroaryl;
R₆ is hydrogen, cyano, nitro, halo, optionally substituted alkyl, or optionally substituted alkoxy;
R₇ is halo, optionally substituted alkyl, cyano, nitro, hydroxy, optionally substituted alkoxy, or optionally substituted amino; and
R₈ is halo, optionally substituted alkyl, or optionally substituted alkoxy;
or R₆ and R₇, together with the carbons to which they are attached, form an optionally substituted 3- to 7-membered ring which optionally includes one or more heteroatoms selected from N, O, and S in the ring; and R₈ is halo, optionally substituted alkyl, or optionally substituted alkoxy;
or R₇ and R₈, together with the carbons to which they are attached, form an optionally substituted 3- to 7-membered ring which optionally includes one or more heteroatoms selected from N, O, and S in the ring; and R₆ is hydrogen, cyano, nitro, halo, optionally substituted alkyl, or optionally substituted alkoxy;
provided that only one of R₆, R₇, and R₈ is optionally substituted alkoxy; and
provided that when R₁ and R₅ are hydrogen, R₂ is methyl, R₃ is methoxy or ethoxy, R₇ is hydroxy, and R₈ is methoxy, then R₆ is not hydrogen or halo.

The compounds of Formula I can be named and numbered in the manner (e.g., using ChemDraw Ultra, Version 8.0, Cambridgesoft Corp., Cambridge, Mass.) described below. For example, the compound: 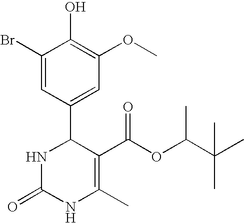
i.e., the compound according to Formula I where R₁ is hydrogen, R₂ is methyl, R₃ is 3,3-dimethylbutan-2-yl, R₅ is hydrogen, R₆ is bromo, R₇ is hydroxy, and R₈ is methoxy can be named 3,3-dimethylbutan-2-yl 4-(3-bromo-4-hydroxy-5-methoxyphenyl)-1,2,3,4-tetrahydro-6-methyl-2-oxopyrimidine-5-carboxylate.

Likewise, the compound: 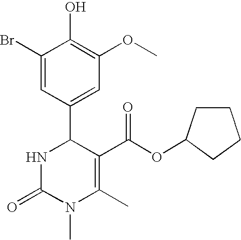
i.e., the compound according to Formula I where R₁ is methyl, R₂ is methyl, R₃ is cyclopentyl, R₅ is hydrogen, R₆ is bromo, R₇ is hydroxy, and R₈ is methoxy can be named cyclopentyl 4-(3-bromo-4-hydroxy-5-methoxyphenyl)-1,2,3,4-tetrahydro-1,6-dimethyl-2-oxopyrimidine-5-carboxylate.

Unless specified otherwise, the terms “solvent”, “inert organic solvent” or “inert solvent” mean a solvent inert under the conditions of the reaction being described in conjunction therewith [including, for example, benzene, toluene, acetonitrile, tetrahydrofuran (“THF”), dimethylformamide (“DMF”), chloroform, methylene chloride (or dichloromethane), diethyl ether, methanol, pyridine and the like]. Unless specified to the contrary, the solvents used in the reactions of the present invention are inert organic solvents.

In general, esters of carboxylic acids may be prepared by conventional esterification procedures, for example alkyl esters may be prepared by treating the required carboxylic acid with the appropriate alkanol, generally under acidic conditions. Likewise, amides may be prepared using conventional amidation procedures, for example amides may be prepared by treating an activated carboxylic acid with the appropriate amine. Alternatively, a lower alkyl ester such as a methyl ester of the acid may be treated with an amine to provide the required amide, optionally in presence of trimethylalluminium following the procedure described in Tetrahedron Lett. 48, 4171-4173, (1977). Carboxyl groups may be protected as alkyl esters, for example methyl esters, which esters may be prepared and removed using conventional procedures, one convenient method for converting carbomethoxy to carboxyl is to use aqueous lithium hydroxide.

The salts and solvates of the compounds mentioned herein may as required be produced by other methods conventional in the art. For example, if an inventive compound is an acid, a desired base addition salt can be prepared by treatment of the free acid with an inorganic or organic base, such as an amine (primary, secondary, or tertiary); an alkali metal or alkaline earth metal hydroxide; or the like. Illustrative examples of suitable salts include organic salts derived from amino acids such as glycine and arginine; ammonia; primary, secondary, and tertiary amines; such as ethylenediamine, and cyclic amines, such as cyclohexylamine, piperidine, morpholine, and piperazine; as well as inorganic salts derived from sodium, calcium, potassium, magnesium, manganese, iron, copper, zinc, aluminum, and lithium.

If a compound is a base, a desired acid addition salt may be prepared by any suitable method known in the art, including treatment of the free base with an organic acid, such as hydrochloric acid, hydrobromic acid, sulfuric acid, nitric acid, phosphoric acid, and the like, or with an organic acid, such as acetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartartic acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid, or the like.

Isolation and purification of the compounds and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples hereinbelow. However, other equivalent separation or isolation procedures can, of course, also be used.

The compounds of Formula I can be prepared using the Biginelli reaction as outlined below: 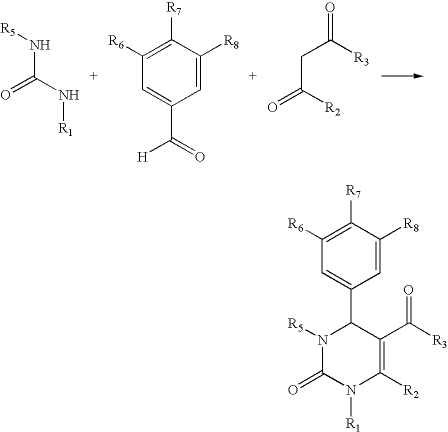
The Biginelli reaction is a well-known method of synthesizing tetrahydropyrimidinones in a one-pot acid-catalyzed condensation of an aldehyde, β-ketoester, and a urea. See, also, Wipf et al. (1995) Tetrahedron Lett., 36(43): 7819-7822; Folkers (1933) J. Am. Chem. Soc., 55, 3784; Fissekis et al. (1973) J. Am. Chem. Soc., 95, 8741; Atwal et al. (1991) J. Med. Chem 34, 806; Atwal (199) J. Med. Chem 33, 2629; H. Cho (1989) J. Med. Chem 32, 2399; Baldwin et al., U.S. Pat. No. 4,675,321, issued Jun. 23, 1987; WO 96/14846; U.S. Pat. No. 5,786,472; and U.S. Patent Application No. 20030008888; each of which is incorporated herein by reference for all purposes.

A compound of Formula I is optionally contacted with a pharmaceutically acceptable acid or base to form the corresponding acid or base addition salt.

A pharmaceutically acceptable acid addition salt of a compound of Formula I is optionally contacted with a base to form the corresponding free base of Formula I.

A pharmaceutically acceptable base addition salt of a compound of Formula I is optionally contacted with an acid to form the corresponding free acid of Formula I.

When considering the compounds of Formula I, in some embodiments, R₁ and R₅ are independently selected from the group consisting of hydrogen, optionally substituted lower alkyl (especially benzyl), and optionally substituted aryl (especially phenyl). In some embodiments, R₁ and R₅ are independently hydrogen or optionally substituted lower alkyl. In some embodiments, R₁ is hydrogen, methyl, ethyl, benzyl, 2-(N,N-dimethylamino)ethyl, carboxymethyl, (ethyoxy)carbonylmethyl, or (2-methoxyethylcarbamoyl)methyl. In some embodiments, R₁ is hydrogen, methyl, or ethyl. In some embodiments, R₁ is hydrogen. In certain embodiments, R₅ is hydrogen. In some embodiments, R₁ and R₅ are hydrogen.

When considering the compounds of Formula I, in some embodiments, R₂ is hydrogen, optionally substituted lower alkyl, optionally substituted aryl (especially phenyl), or optionally substituted heteroaryl. In some embodiments, R₂ is optionally substituted lower alkyl (especially optionally substituted methyl). In some embodiments, R₂ is methyl, halomethyl, alkoxycarbonylmethyl-, carboxymethyl-, alkoxymethyl, or hydroxymethyl. In certain embodiments, R₂ is methyl.

When considering the compounds of Formula I, in some embodiments R₃ is optionally substituted alkoxy, optionally substituted alkyl, or NR₉R₁₀ wherein R₉ and R₁₀ are independently selected from hydrogen, optionally substituted lower alkyl, optionally substituted aryl or optionally substituted heteroaryl. In some embodiments R₃ is optionally substituted alkoxy. In some embodiments, R₃ is optionally substituted C₁-C₈ alkoxy. In some embodiments, R₃ is methoxy-, ethoxy-, propoxy-, isopropoxy-, butoxy-, pentoxy-, c-pentoxy-, hexoxy-, c-hexyloxy-, heptoxy, c-heptoxy-, 1,2,2-trimethylpropoxy-, 1,1,1-trifluoro-isopropoxy-, 1-methyl-propoxy-, 2-methyl-propoxy-, 3-methyl-butoxy-, t-butoxy-, benzyloxy-, 4-methyl-benzyloxy-, or 1-(2-isopropoxyethoxy).

When considering the compounds of Formula I, in some embodiments R₃ is NR₉R₁₀ wherein R₉ and R₁₀ are independently selected from hydrogen, optionally substituted lower alkyl, optionally substituted aryl or optionally substituted heteroaryl. In some embodiments R₃ is NR₉R₁₀ wherein R₉ and R₁₀ are independently selected from hydrogen or optionally substituted lower alkyl.

In some embodiments, R₃ is optionally substituted alkyl. In some embodiments, R₃ is optionally substituted lower alkyl.

When considering the compounds of Formula I, in some embodiments, R₆ is hydrogen, cyano, nitro, halo, optionally substituted alkyl, or optionally substituted alkoxy. In some embodiments, R₆ is hydrogen, bromo, chloro, fluoro, methyl, trifluoromethyl, hydroxymethyl, or methoxy.

When considering the compounds of Formula I, in some embodiments, R₇ is halo, optionally substituted alkyl, cyano, nitro, hydroxy, optionally substituted alkoxy, or optionally substituted amino. In some embodiments, R₇ is hydroxy, optionally substituted alkoxy, or optionally substituted amino. In certain embodiments, R₇ is hydroxy. In some embodiments, R₇ is optionally substituted lower alkoxy. In some embodiments, R₇ is benzyloxy or methoxy. In some embodiments, R₇ is methoxy. In some embodiments, R₇ is acetylamino or amino.

When considering the compounds of Formula I, in some embodiments, R₈ is halo, optionally substituted alkyl, or optionally substituted alkoxy. In some embodiments, R₈ is bromo, chloro, fluoro, methyl, trifluoromethyl, hydroxymethyl, or methoxy.

In some embodiments, R₆ and R₇, together with the carbons to which they are attached, form an optionally substituted 5- or 6-membered ring which includes one or more (such as one, two, or three, for example, one or two) heteroatoms selected from N, O, and S in the ring and and R₈ is halo, optionally substituted alkyl, or optionally substituted alkoxy. In some embodiments, R₆ and R₇, together with the phenyl ring to which they are attached, form a substituted 1H-indole, 2,3-dihydro-benzo[1,4]dioxine, substituted benzimidazole, or substituted benzo[1,3]dioxole.

In some embodiments, R₇ and R₈, together with the carbons to which they are attached, form an optionally substituted 3- to 7-membered ring which optionally includes one or more (such as one, two, or three, for example, one or two) heteroatoms selected from N, O, and S in the ring; and R₆ is hydrogen, cyano, nitro, halo, optionally substituted alkyl, or optionally substituted alkoxy. In some embodiments, R₇ and R₈, together with the phenyl ring to which they are attached, form a substituted 1H-indole, 2,3-dihydro-benzo[1,4]dioxine, substituted benzimidazole, or substituted benzo[1,3]dioxole.

In some embodiments, at least one of R₆ and R₈ is halo. In some embodiments, at least one of R₆ and R₈ is optionally substituted alkoxy. In some embodiments, at least one of R₆ and R₈ is trifluoromethyl. In some embodiments, at least one of R₆ and R₈ is methyl.

In some embodiments,
R₁ and R₅ are independently hydrogen or optionally substituted lower alkyl (especially, R₁ is methyl, ethyl, benzyl, (ethyoxy)carbonylmethyl, and (2-methoxyethylcarbamoyl)methyl and R₅ is hydrogen);
R₂ is optionally substituted lower alkyl (especially optionally substituted methyl);
R₃ is optionally substituted alkoxy or optionally substituted alkyl;
R₆ is hydrogen, cyano, nitro, halo, optionally substituted alkyl, or optionally substituted alkoxy;
R₇ is halo, optionally substituted alkyl, cyano, nitro, hydroxy, optionally substituted alkoxy, or optionally substituted amino; and
R₈ is halo, optionally substituted alkyl, or optionally substituted alkoxy, provided that only one of R₆, R₇, and R₈ is optionally substituted alkoxy; and provided that when R₁ and R₅ are hydrogen, R₂ is methyl, R₃ is methoxy or ethoxy, R₇ is hydroxy, and R₈ is methoxy, then R₆ is not hydrogen or halo.

In some embodiments,
R₁ and R₅ are independently hydrogen or optionally substituted lower alkyl (especially, R₁ is methyl, ethyl, benzyl, (ethyoxy)carbonylmethyl, and (2-methoxyethylcarbamoyl)methyl and R₅ is hydrogen);
R₂ is optionally substituted lower alkyl (especially optionally substituted methyl);
R₃ is optionally substituted alkoxy or optionally substituted alkyl;
R₆ is hydrogen, bromo, chloro, fluoro, methyl, trifluoromethyl, or methoxy;
R₇ is halo, optionally substituted alkyl, cyano, nitro, hydroxy, optionally substituted alkoxy, or optionally substituted amino; and
R₈ is halo, optionally substituted alkyl, or optionally substituted alkoxy, provided that only one of R₆, R₇, and R₈ is optionally substituted alkoxy; and provided that when R₁ and R₅ are hydrogen, R₂ is methyl, R₃ is methoxy or ethoxy, R₇ is hydroxy, and R₈ is methoxy, then R₆ is not hydrogen or halo.

In some embodiments,
R₁ and R₅ are independently hydrogen or optionally substituted lower alkyl, (especially, R₁ is methyl, ethyl, benzyl, (ethyoxy)carbonylmethyl, and (2-methoxyethylcarbamoyl)methyl and R₅ is hydrogen);
R₂ is optionally substituted lower alkyl (especially optionally substituted methyl);
R₃ is optionally substituted alkoxy or optionally substituted alkyl;
R₆ is hydrogen, cyano, nitro, halo, optionally substituted alkyl, or optionally substituted alkoxy;
R₇ is hydroxy; and
R₈ is halo, optionally substituted alkyl, or optionally substituted alkoxy, provided that only one of R₆, R₇, and R₈ is optionally substituted alkoxy; and provided that when R₁ and R₅ are hydrogen, R₂ is methyl, R₃ is methoxy or ethoxy, R₇ is hydroxy, and R₈ is methoxy, then R₆ is not hydrogen or halo.

In some embodiments,
R₁ and R₅ are independently hydrogen or optionally substituted lower alkyl, (especially, R₁ is methyl, ethyl, benzyl, (ethyoxy)carbonylmethyl, and (2-methoxyethylcarbamoyl)methyl and R₅ is hydrogen);
R₂ is optionally substituted lower alkyl (especially optionally substituted methyl);
R₃ is optionally substituted alkoxy or optionally substituted alkyl;
R₆ is hydrogen, cyano, nitro, halo, optionally substituted alkyl, or optionally substituted alkoxy;
R₇ is methoxy; and
R₈ is halo, optionally substituted alkyl, or optionally substituted alkoxy, provided that only one of R₆, R₇, and R₈ is optionally substituted alkoxy; and provided that when R₁ and R₅ are hydrogen, R₂ is methyl, R₃ is methoxy or ethoxy, R₇ is hydroxy, and R₈ is methoxy, then R₆ is not hydrogen or halo.

In some embodiments,
R₁ and R₅ are independently hydrogen or optionally substituted lower alkyl, (especially, R₁ is methyl, ethyl, benzyl, (ethyoxy)carbonylmethyl, and (2-methoxyethylcarbamoyl)methyl and R₅ is hydrogen);
R₂ is optionally substituted lower alkyl (especially optionally substituted methyl);
R₃ is optionally substituted alkoxy or optionally substituted alkyl;
R₆ is hydrogen, cyano, nitro, halo, optionally substituted alkyl, or optionally substituted alkoxy;
R₇ is amino; and
R₈ is halo, optionally substituted alkyl, or optionally substituted alkoxy, provided that only one of R₆, R₇, and R₈ is optionally substituted alkoxy; and provided that when R₁ and R₅ are hydrogen, R₂ is methyl, R₃ is methoxy or ethoxy, R₇ is hydroxy, and R₈ is methoxy, then R₆ is not hydrogen or halo.

In some embodiments,
R₁ and R₅ are independently hydrogen or optionally substituted lower alkyl, (especially, R₁ is methyl, ethyl, benzyl, (ethyoxy)carbonylmethyl, and (2-methoxyethylcarbamoyl)methyl and R₅ is hydrogen);
R₂ is optionally substituted lower alkyl (especially optionally substituted methyl);
R₃ is optionally substituted alkoxy or optionally substituted alkyl;
R₆ is hydrogen, cyano, nitro, halo, optionally substituted alkyl, or optionally substituted alkoxy;
R₇ is halo, optionally substituted alkyl, cyano, nitro, hydroxy, optionally substituted alkoxy, or optionally substituted amino; and
R₈ is bromo, chloro, fluoro, methyl, trifluoromethyl, or methoxy, provided that only one of R₆, R₇, and R₈ is optionally substituted alkoxy; and provided that when R₁ and R₅ are hydrogen, R₂ is methyl, R₃ is methoxy or ethoxy, R₇ is hydroxy, and R₈ is methoxy, then R₆ is not hydrogen or halo.

In some embodiments,
R₁ and R₅ are independently hydrogen or optionally substituted lower alkyl, (especially, R₁ is methyl, ethyl, benzyl, (ethyoxy)carbonylmethyl, and (2-methoxyethylcarbamoyl)methyl and R₅ is hydrogen);
R₂ is optionally substituted lower alkyl (especially optionally substituted methyl);
R₃ is optionally substituted alkoxy or optionally substituted alkyl;
R₆ is hydrogen, bromo, chloro, fluoro, methyl, trifluoromethyl, or methoxy;
R₇ is hydroxy; and
R₈ is bromo, chloro, fluoro, methyl, trifluoromethyl, or methoxy, provided that only one of R₆, R₇, and R₈ is optionally substituted alkoxy; and provided that when R₁ and R₅ are hydrogen, R₂ is methyl, R₃ is methoxy or ethoxy, R₇ is hydroxy, and R₈ is methoxy, then R₆ is not hydrogen or halo.

In some embodiments,
R₁ and R₅ are independently hydrogen or optionally substituted lower alkyl, (especially, R₁ is methyl, ethyl, benzyl, (ethyoxy)carbonylmethyl, and (2-methoxyethylcarbamoyl)methyl and R₅ is hydrogen);
R₂ is optionally substituted lower alkyl (especially optionally substituted methyl);
R₃ is optionally substituted alkoxy or optionally substituted alkyl;
R₆ is hydrogen, bromo, chloro, fluoro, methyl, trifluoromethyl, or methoxy;
R₇ is methoxy; and
R₈ is bromo, chloro, fluoro, methyl, trifluoromethyl, or methoxy, provided that only one of R₆, R₇, and R₈ is optionally substituted alkoxy; and provided that when R₁ and R₅ are hydrogen, R₂ is methyl, R₃ is methoxy or ethoxy, R₇ is hydroxy, and R₈ is methoxy, then R₆ is not hydrogen or halo.

In some embodiments,
R₁ and R₅ are independently hydrogen or optionally substituted lower alkyl, (especially, R₁ is methyl, ethyl, benzyl, (ethyoxy)carbonylmethyl, and (2-methoxyethylcarbamoyl)methyl and R₅ is hydrogen);
R₂ is optionally substituted lower alkyl (especially optionally substituted methyl);
R₃ is optionally substituted alkoxy or optionally substituted alkyl;
R₆ is hydrogen, bromo, chloro, fluoro, methyl, trifluoromethyl, or methoxy;
R₇ is amino; and
R₈ is bromo, chloro, fluoro, methyl, trifluoromethyl, or methoxy, provided that only one of R₆, R₇, and R₈ is optionally substituted alkoxy; and provided that when R₁ and R₅ are hydrogen, R₂ is methyl, R₃ is methoxy or ethoxy, R₇ is hydroxy, and R₈ is methoxy, then R₆ is not hydrogen or halo.

In some embodiments,
R₁ and R₅ are independently hydrogen or optionally substituted lower alkyl, (especially, R₁ is methyl, ethyl, benzyl, (ethyoxy)carbonylmethyl, and (2-methoxyethylcarbamoyl)methyl and R₅ is hydrogen);
R₂ is optionally substituted lower alkyl (especially optionally substituted methyl);
R₃ is optionally substituted alkoxy or optionally substituted alkyl;
R₆ is hydrogen, cyano, nitro, halo, optionally substituted alkyl, or optionally substituted alkoxy;
R₇ is halo, optionally substituted alkyl, cyano, nitro, hydroxy, optionally substituted alkoxy, or optionally substituted amino; and
R₈ is halo, optionally substituted alkyl, or optionally substituted alkoxy, provided that at least one of R₆ and R₈ is halo, provided that only one of R₆, R₇, and R₈ is optionally substituted alkoxy; and provided that when R₁ and R₅ are hydrogen, R₂ is methyl, R₃ is methoxy or ethoxy, R₇ is hydroxy, and R₈ is methoxy, then R₆ is not hydrogen or halo.

In some embodiments,
R₁ and R₅ are independently hydrogen or optionally substituted lower alkyl, (especially, R₁ is methyl, ethyl, benzyl, (ethyoxy)carbonylmethyl, and (2-methoxyethylcarbamoyl)methyl and R₅ is hydrogen);
R₂ is optionally substituted lower alkyl (especially optionally substituted methyl);
R₃ is optionally substituted alkoxy or optionally substituted alkyl;
R₆ is hydrogen, cyano, nitro, halo, optionally substituted alkyl, or optionally substituted alkoxy;
R₇ is halo, optionally substituted alkyl, cyano, nitro, hydroxy, optionally substituted alkoxy, or optionally substituted amino; and
R₈ is halo, optionally substituted alkyl, or optionally substituted alkoxy; provided that at least one of R₆ and R₈ is optionally substituted alkoxy, provided that only one of R₆, R₇, and R₈ is optionally substituted alkoxy; and provided that when R₁ and R₅ are hydrogen, R₂ is methyl, R₃ is methoxy or ethoxy, R₇ is hydroxy, and R₈ is methoxy, then R₆ is not hydrogen or halo.

Particular compounds of the invention are:
3,3-dimethylbutan-2-yl 4-(3-bromo-4-hydroxy-5-methoxyphenyl)-1,2,3,4-tetrahydro-6-methyl-2-oxopyrimidine-5-carboxylate
cyclopentyl 4-(3-bromo-4-hydroxy-5-methoxyphenyl)-1,2,3,4-tetrahydro-1,6-dimethyl-2-oxopyrimidine-5-carboxylate
3,3-dimethylbutan-2-yl 4-(3-chloro-4-hydroxy-5-methoxyphenyl)-1,2,3,4-tetrahydro-6-methyl-2-oxopyrimidine-5-carboxylate
sec-butyl 4-(3-bromo-4-hydroxy-5-methoxyphenyl)-1,2,3,4-tetrahydro-6-methyl-2-oxopyrimidine-5-carboxylate
isopentyl 4-(3-bromo-4-hydroxy-5-methoxyphenyl)-1,2,3,4-tetrahydro-1,6-dimethyl-2-oxopyrimidine-5-carboxylate
ethyl 4-(3-bromo-4-methoxyphenyl)-1,2,3,4-tetrahydro-6-methyl-2-oxopyrimidine-5-carboxylate
isobutyl 4-(3-chloro-4-hydroxy-5-methoxyphenyl)-1,2,3,4-tetrahydro-6-methyl-2-oxopyrimidine-5-carboxylate
cyclopentyl 4-(3-chloro-4-hydroxy-5-methoxyphenyl)-1,2,3,4-tetrahydro-1,6-dimethyl-2-oxopyrimidine-5-carboxylate
sec-butyl 4-(3-chloro-4-hydroxy-5-methoxyphenyl)-1,2,3,4-tetrahydro-6-methyl-2-oxopyrimidine-5-carboxylate
ethyl 4-(benzo[d][1,3]dioxol-5-yl)-6-(chloromethyl)-1,2,3,4-tetrahydro-2-oxopyrimidine-5-carboxylate
3,3-dimethylbutan-2-yl 1,2,3,4-tetrahydro-4-(4-hydroxy-3-methoxy-5-methylphenyl)-6-methyl-2-oxopyrimidine-5-carboxylate
3,3-dimethylbutan-2-yl 1-((ethoxycarbonyl)methyl)-4-(3-bromo-4-hydroxy-5-methoxyphenyl)-1,2,3,4-tetrahydro-6-methyl-2-oxopyrimidine-5-carboxylate
3,3-dimethylbutan-2-yl 4-(3-bromo-4-hydroxy-5-methoxyphenyl)-1-ethyl-1,2,3,4-tetrahydro-6-methyl-2-oxopyrimidine-5-carboxylate
cyclopentyl 4-(3-chloro-4-hydroxy-5-methoxyphenyl)-1,2,3,4-tetrahydro-1,6-dimethyl-2-oxopyrimidine-5-carboxylate
tert-butyl 4-(3-chloro-4-hydroxy-5-methoxyphenyl)-1,2,3,4-tetrahydro-6-methyl-2-oxopyrimidine-5-carboxylate
cyclohexyl 4-(3-bromo-4-hydroxy-5-methoxyphenyl)-1,2,3,4-tetrahydro-6-methyl-2-oxopyrimidine-5-carboxylate
sec-butyl 4-(3-chloro-4-hydroxy-5-methoxyphenyl)-1,2,3,4-tetrahydro-6-methyl-2-oxopyrimidine-5-carboxylate
tert-butyl 1,2,3,4-tetrahydro-4-(4-hydroxy-3-methoxyphenyl)-6-methyl-2-oxopyrimidine-5-carboxylate
4,4-dimethylpentan-2-yl 1-((2-methoxyethylcarbamoyl)methyl)-4-(3-bromo-4-hydroxy-5-methoxyphenyl)-1,2,3,4-tetrahydro-6-methyl-2-oxopyrimidine-5-carboxylate
tert-butyl 1-((ethoxycarbonyl)methyl)-4-(3-bromo-4-hydroxy-5-methoxyphenyl)-1,2,3,4-tetrahydro-6-methyl-2-oxopyrimidine-5-carboxylate
tert-butyl 1-benzyl-4-(3-bromo-4-hydroxy-5-methoxyphenyl)-1,2,3,4-tetrahydro-6-methyl-2-oxopyrimidine-5-carboxylate
cycloheptyl 4-(3-chloro-4-hydroxy-5-methoxyphenyl)-1,2,3,4-tetrahydro-6-methyl-2-oxopyrimidine-5-carboxylate
tert-butyl 1,2,3,4-tetrahydro-4-(1H-indol-5-yl)-6-methyl-2-oxopyrimidine-5-carboxylate
sec-butyl 4-(3-bromo-4-hydroxy-5-methoxyphenyl)-1,2,3,4-tetrahydro-6-methyl-2-oxopyrimidine-5-carboxylate
3,3-dimethylbutan-2-yl 4-(3-fluoro-4-hydroxy-5-methoxyphenyl)-1,2,3,4-tetrahydro-6-methyl-2-oxopyrimidine-5-carboxylate
tert-butyl 1,2,3,4-tetrahydro-4-(4-hydroxy-3-methoxy-5-methylphenyl)-6-methyl-2-oxopyrimidine-5-carboxylate
tert-butyl 4-(3-bromo-5-ethoxy-4-hydroxyphenyl)-1,2,3,4-tetrahydro-6-methyl-2-oxopyrimidine-5-carboxylate
ethyl 4-(3-bromo-4-methoxyphenyl)-1,2,3,4-tetrahydro-6-methyl-2-oxopyrimidine-5-carboxylate
tert-butyl 4-(3-bromo-4-hydroxy-5-methoxyphenyl)-1,2,3,4-tetrahydro-1,6-dimethyl-2-oxopyrimidine-5-carboxylate
3,3-dimethylbutan-2-yl 1,2,3,4-tetrahydro-4-(4-hydroxy-3-methoxyphenyl)-6-methyl-2-oxopyrimidine-5-carboxylate
isopropyl 4-(3-chloro-4-hydroxy-5-methoxyphenyl)-1,2,3,4-tetrahydro-6-methyl-2-oxopyrimidine-5-carboxylate
tert-butyl 4-(3-bromo-4-hydroxy-5-methoxyphenyl)-1,2,3,4-tetrahydro-6-methyl-2-oxopyrimidine-5-carboxylate
tert-butyl 4-(3-fluoro-4-hydroxy-5-methoxyphenyl)-1,2,3,4-tetrahydro-6-methyl-2-oxopyrimidine-5-carboxylate
tert-butyl 4-(3-bromo-4-hydroxyphenyl)-1,2,3,4-tetrahydro-6-methyl-2-oxopyrimidine-5-carboxylate
4-(3-bromo-4-hydroxy-5-methoxy-phenyl)-6-methyl-2-oxo-1,2,3,4-tetrahydro-pyrimidine-5-carboxylic acid 1,2,2-trimethyl-propyl ester
4-(3-Bromo-4-hydroxy-5-methoxy-phenyl)-6-methyl-2-oxo-1,2,3,4-tetrahydro-pyrimidine-5-carboxylic acid isopropyl ester
4-(3-Bromo-4-hydroxy-5-methoxy-phenyl)-1-carboxymethyl-6-methyl-2-oxo-1,2,3,4-tetrahydro-pyrimidine-5-carboxylic acid 1,2,2-trimethyl-propyl ester
4-(4-Hydroxy-3-methoxy-5-methyl-phenyl)-6-methyl-2-oxo-1,2,3,4-tetrahydro-pyrimidine-5-carboxylic acid 1,2,2-trimethyl-propyl ester
4-(2-Bromo-phenyl)-6-methyl-2-oxo-1,2,3,4-tetrahydro-pyrimidine-5-carboxylic acid sec-butyl ester
4-(3,5-Dichloro-4-hydroxy-phenyl)-6-methyl-2-oxo-1,2,3,4-tetrahydro-pyrimidine-5-carboxylic acid tert-butyl ester
4-(3,5-Dimethoxy-4-hydroxy-phenyl)-6-methyl-2-oxo-1,2,3,4-tetrahydro-pyrimidine-5-carboxylic acid tert-butyl ester
4-(4-Hydroxy-3-methoxy-5-nitro-phenyl)-6-methyl-2-oxo-1,2,3,4-tetrahydro-pyrimidine-5-carboxylic acid tert-butyl ester
4-(4-Fluoro-3-methoxy-phenyl)-6-methyl-2-oxo-1,2,3,4-tetrahydro-pyrimidine-5-carboxylic acid tert-butyl ester
4-(4-Acetylamino-3,5-dibromo-phenyl)-6-methyl-2-oxo-1,2,3,4-tetrahydro-pyrimidine-5-carboxylic acid tert-butyl ester
4-(3-Bromo-4-hydroxy-5-methoxy-phenyl)-1,3,6-trimethyl-2-oxo-1,2,3,4-tetrahydro-pyrimidine-5-carboxylic acid tert-butyl ester
4-(4-Hydroxy-3-methoxy-5-cyano-phenyl)-6-methyl-2-oxo-1,2,3,4-tetrahydro-pyrimidine-5-carboxylic acid 1,2,2-trimethyl-propyl ester
4-(4-Hydroxy-3-methoxy-5-(hydroxymethyl)-phenyl)-6-methyl-2-oxo-1,2,3,4-tetrahydro-pyrimidine-5-carboxylic acid 1,2,2-trimethyl-propyl ester
4-(4-Hydroxy-3,5-dimethyl-phenyl)-6-methyl-2-oxo-1,2,3,4-tetrahydro-pyrimidine-5-carboxylic acid 1,2,2-trimethyl-propyl ester
4-(1H-Benzoimidazol-5-yl)-6-methyl-2-oxo-1,2,3,4-tetrahydro-pyrimidine-5-carboxylic acid 1,2,2-trimethyl-propyl ester
6-Methyl-4-(7-methyl-1H-benzoimidazol-5-yl)-2-oxo-1,2,3,4-tetrahydro-pyrimidine-5-carboxylic acid 1,2,2-trimethyl-propyl ester
6-Methyl-4-(7-methoxy-1H-indol-5-yl)-2-oxo-1,2,3,4-tetrahydro-pyrimidine-5-carboxylic acid 1,2,2-trimethyl-propyl ester
6-Methyl-4-(7-methyl-1H-indol-5-yl)-2-oxo-1,2,3,4-tetrahydro-pyrimidine-5-carboxylic acid 1,2,2-trimethyl-propyl ester
4-(4-Benzyloxy-3-methoxy-5-methyl-phenyl)-6-methyl-2-oxo-1,2,3,4-tetrahydro-pyrimidine-5-carboxylic acid 1,2,2-trimethyl-propyl ester
1-Ethyl-4-(4-hydroxy-3-methoxy-5-methyl-phenyl)-6-methyl-2-oxo-1,2,3,4-tetrahydro-pyrimidine-5-carboxylic acid 1,2,2-trimethyl-propyl ester
1-(2-Dimethylamino-ethyl)-4-(4-hydroxy-3-methoxy-5-methyl-phenyl)-6-methyl-2-oxo-1,2,3,4-tetrahydro-pyrimidine-5-carboxylic acid 1,2,2-trimethyl-propyl ester
1-Ethoxycarbonylmethyl-4-(4-hydroxy-3-methoxy-5-methyl-phenyl)-6-methyl-2-oxo-1,2,3,4-tetrahydro-pyrimidine-5-carboxylic acid 1,2,2-trimethyl-propyl ester
4-(3-Bromo-4-hydroxy-5-methoxy-phenyl)-1-[(2-methoxy-ethylcarbamoyl)-methyl]-6-methyl-2-oxo-1,2,3,4-tetrahydro-pyrimidine-5-carboxylic acid 1,2,2-trimethyl-propyl ester
4-(3-Cyano-4-hydroxy-5-methoxy-phenyl)-6-methyl-2-oxo-1,2,3,4-tetrahydro-pyrimidine-5-carboxylic acid 1,2,2-trimethyl-propyl ester
4-(3-Bromo-4-hydroxy-5-methoxy-phenyl)-6-methyl-2-oxo-1,2,3,4-tetrahydro-pyrimidine-5-carboxylic acid 1,2,2-trimethyl-propyl ester
4-(3-Bromo-4-hydroxy-5-methoxy-phenyl)-6-methyl-2-oxo-1,2,3,4-tetrahydro-pyrimidine-5-carboxylic acid cyclopentyl ester

Additional compounds of the invention include:
p-tolyl-1,2,3,4-tetrahydro-4-(3,4,5-trimethoxyphenyl)-6-methyl-2-oxopyrimidine-5-carboxylate
tert-butyl 4-(4-amino-3,5-dibromophenyl)-1,2,3,4-tetrahydro-6-methyl-2-oxopyrimidine-5-carboxylate
2-isopropoxyethyl1,2,3,4-tetrahydro-4-(3,4,5-trimethoxyphenyl)-6-methyl-2-oxopyrimidine-5-carboxylate tert-butyl 4-(3-chloro-4,5-dimethoxyphenyl)-1,2,3,4-tetrahydro-6-methyl-2-oxopyrimidine-5-carboxylate.

Compounds of the invention will generally be capable of forming acid addition salts (i.e., will comprise a site which reacts with a pharmaceutically acceptable acid to form an acid addition salt.) The present invention includes pharmaceutically acceptable acid addition salts of the compounds of Formula I. Acid addition salts of the present compounds are prepared in a standard manner in a suitable solvent from the parent compound and an excess of an acid, such as hydrochloric, hydrobromic, sulfuric, phosphoric, acetic, maleic, succinic or methanesulfonic.

The salts and/or solvates of the compounds of Formula I which are not pharmaceutically acceptable may be useful as intermediates in the preparation of pharmaceutically acceptable salts and/or solvates of compounds of Formula I or the compounds of Formula I themselves, and as such form another aspect of the present invention.

The compounds of the invention are used to treat cellular proliferation diseases. Such disease states which can be treated by the compounds, compositions and methods provided herein include, but are not limited to, cancer (further discussed below), autoimmune disease, fungal disorders, arthritis, graft rejection, inflammatory bowel disease, cellular proliferation induced after medical procedures, including, but not limited to, surgery, angioplasty, and the like. Treatment includes inhibiting cellular proliferation. It is appreciated that in some cases the cells may not be in an abnormal state and still require treatment. Thus, in one embodiment, the invention herein includes application to cells or individuals afflicted or subject to impending affliction with any one of these disorders or states.

The compounds, pharmaceutical formulations and methods provided herein are particularly deemed useful for the treatment of cancer including solid tumors such as skin, breast, brain, cervical carcinomas, testicular carcinomas, etc. More particularly, cancers that can be treated include, but are not limited to:
Cardiac: sarcoma (angiosarcoma, fibrosarcoma, rhabdomyosarcoma, liposarcoma), myxoma, rhabdomyoma, fibroma, lipoma and teratoma;
Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;
Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma);
Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);
Liver: hepatoma (hepatocellular carcinoma), cholangiocarcinoma, hepatoblastoma, angiosarcoma, hepatocellular adenoma, hemangioma;
Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors;
Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma);
Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant tertoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma], fallopian tubes (carcinoma);
Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma];
Skin: malignant melanoma, basal cell carcinoma, squamous cell carcinoma, Karposi's sarcoma, moles dysplastic nevi, lipoma, angioma, dermatofibroma, keloids, psoriasis; and
Adrenal glands: neuroblastoma.
   As used herein, treatment of cancer includes treatment of cancerous cells, including cells afflicted by any one of the above-identified conditions. Thus, the term “cancerous cell” as provided herein, includes a cell afflicted by any one of the above identified conditions.

Another useful aspect of the invention is a kit having a compound, salt or solvate of Formula I and a package insert or other labeling including directions treating a cellular proliferative disease by administering an effective amount of the compound, salt or solvate. The compound, salt or solvate of Formula I in the kits of the invention is particularly provided as one or more doses for a course of treatment for a cellular proliferative disease, each dose being a pharmaceutical formulation including a pharmaceutical excipient and a compound, salt or solvate of Formula 1.

The compounds of the invention may be demonstrated to inhibit tumor cell proliferation, cell transformation and tumorigenesis in vitro and in vivo using a variety of assays known in the art, or described herein. Such assays may use cells of a cancer cell line, or cells from a patient. Many assays well-known in the art can be used to assess such survival and/or growth; for example, cell proliferation can be assayed by measuring ³thymidine incorporation, by direct cell count, by detecting changes in transcription, translation or activity of known genes such as proto-oncogenes (e.g., fos, myc) or cell cycle markers (Rb, cdc2, cyclin A, D1, D2, D3, E, etc). The levels of such protein and mRNA and activity can be determined by any method well known in the art.

The present invention provides for cell cycle and cell proliferation analysis by a variety of techniques known in the art. For example, Cell proliferation may be measured by counting samples of a cell population over time (e.g. daily cell counts). Cells may be counted using a hemacytometer and light microscopy (e.g. HyLite hemacytometer, Hausser Scientific). Cell number may be plotted against time in order to obtain a growth curve for the population of interest. In one embodiment, cells are first mixed with the dye Trypan-blue (Sigma), such that living cells exclude the dye, and are counted as viable members of the population.

DNA content and/or mitotic index of the cells may be measured, for example, based on DNA ploidy value of the cell. For example, cells in the G1 phase of the cell cycle generally contain a 2N DNA ploidy value. Cells in which DNA has been replicated but have not progressed through mitosis (e.g. cells in S-phase) will exhibit a ploidy value higher than 2N and up to 4N DNA content. Ploidy value and cell-cycle kinetics may be further measured using propidum iodide assay (see e.g. Turner, T., et al., 1998, Prostate 34:175-81). Alternatively, the DNA ploidy maybe determined by quantitation of DNA Feulgen staining (which binds to DNA in a stoichiometric manner) on a computerized microdensitometrystaining system (see e.g., Bacus, S., 1989, Am. J. Pathol. 135:783-92). In an another embodiment, DNA content may be analyzed by preparation of a chromosomal spread (Zabalou, S., 1994, Hereditas. 120:127-40; Pardue, 1994, Meth. Cell Biol. 44:333-351).

Detection of changes in length of the cell cycle or speed of cell cycle may also be used to measure inhibition of cell proliferation by the compounds of the invention. In one embodiment the length of the cell cycle is determined by the doubling time of a population of cells (e.g., using cells contacted or not contacted with one or more compounds of the invention). In another embodiment, FACS analysis is used to analyze the phase of cell cycle progression, or purify G1, S, and G2/M fractions (see e.g., Delia, D. et al., 1997, Oncogene 14:2137-47).

Lapse of cell cycle checkpoint(s), and/or induction of cell cycle checkpoint(s), may be examined by any method known in the art. Without limitation, a cell cycle checkpoint is a mechanism which ensures that a certain cellular events occur in a particular order. Checkpoint genes are defined by mutations that allow late events to occur without prior completion of an early event (Weinert, T., and Hartwell, L., 1993, Genetics, 134:63-80). Induction or inhibition of cell cycle checkpoint genes may be assayed, for example, by Western blot analysis, or by immunostaining, etc. Lapse of cell cycle checkpoints may be further assessed by the progression of a cell through the checkpoint without prior occurrence of specific events (e.g. progression into mitosis without complete replication of the genomic DNA).

The compounds can also be demonstrated to alter cell proliferation in cultured cells in vitro using methods which are well known in the art. Specific examples of cell culture models include, but are not limited to, for lung cancer, primary rat lung tumor cells (Swafford et al., 1997, Mol. Cell. Biol., 17:1366-1374) and large-cell undifferentiated cancer cell lines (Mabry et al., 1991, Cancer Cells, 3:53-58); colorectal cell lines for colon cancer (Park and Gazdar, 1996, J. Cell Biochem. Suppl. 24:131-141); multiple established cell lines for breast cancer (Hambly et al., 1997, Breast Cancer Res. Treat. 43:247-258; Gierthy et al., 1997, Chemosphere 34:1495-1505; Prasad and Church, 1997, Biochem. Biophys. Res. Commun. 232:14-19); a number of well-characterized cell models for prostate cancer (Webber et al., 1996, Prostate, Part 1, 29:386-394; Part 2, 30:58-64; and Part 3, 30-136-142; Boulikas, 1997, Anticancer Res. 17:1471-1505); for genitourinary cancers, continuous human bladder cancer cell lines (Ribeiro et al., 1997, Int. J. Radiat. Biol. 72:11-20); organ cultures of transitional cell carcinomas (Booth et al., 1997, Lab Invest. 76:843-857) and rat progression models (Vet et al., 1997, Biochim. Biophys Acta 1360:39-44); and established cell lines for leukemias and lymphomas (Drexler, 1994, Leuk. Res. 18:919-927, Tohyama, 1997, Int. J. Hematol. 65:309-317).

The compounds can also be demonstrated to inhibit cell growth (or mitosis) in vitro. In this embodiment, cells are contacted with one or more compounds of the invention, and examined for lethal phenotype.

The compounds can also be demonstrated to inhibit tumor formation in vivo. A vast number of animal models of hyperproliferative disorders, including ftumorigenesis and metastatic spread, are known in the art (see Table 317-1, Chapter 317, “Principals of Neoplasia,” in Harrison's Principals of Internal Medicine, 13th Edition, Isselbacher et al., eds., McGraw-Hill, New York, p. 1814, and Lovejoy et al., 1997, J. Pathol. 181:130-135). Specific examples include for lung cancer, transplantation of tumor nodules into rats (Wang et al., 1997, Ann. Thorac. Surg. 64:216-219) or establishment of lung cancer metastases in SCID mice depleted of NK cells (Yono and Sone, 1997, Gan To Kagaku Ryoho 24:489-494); for colon cancer, colon cancer transplantation of human colon cancer cells into nude mice (Gutman and Fidler, 1995, World J. Surg. 19:226-234), the cotton top tamarin model of human ulcerative colitis (Warren, 1996, Aliment. Pharmacol. Ther. Supp 12:45-47) and mouse models with mutations of the adenomatous polyposis tumor suppressor (Polakis, 1997, Biochim. Biophys. Acta 1332:F127-F147); for breast cancer, transgenic models of breast cancer (Dankfort and Muller, 1996, Cancer Treat. Res. 83:71-88; Amundadittir et al., 1996, Breast Cancer Res. Treat. 39:119-135) and chemical induction of tumors in rats (Russo and Russo, 1996, Breast Cancer Res. Treat. 39:7-20); for prostate cancer, chemically-induced and transgenic rodent models, and human xenograft models (Royai et al., 1996, Semin. Oncol. 23:35-40); for genitourinary cancers, induced bladder neoplasm in rats and mice (Oyasu, 1995, Food Chem. Toxicol 33:747-755) and xenografts of human transitional cell carcinomas into nude rats (Jarrett et al., 1995, J. Endourol. 9:1-7); and for hematopoietic cancers, transplanted allogenic marrow in animals (Appelbaum, 1997, Leukemia 11(Suppl. 4):S15-S17). Further, general animal models applicable to many types of cancer have been described, including, but not restricted to, the p53-deficient mouse model (Donehower, 1996, Semin. Cancer Biol. 7:269-278), the Min mouse (Shoemaker et al., 1997, Biochem. Biophys. Acta, 1332:F25-F48), and immune responses to tumors in rat (Frey, 1997, Methods, 12:173-188).

For example, a compound can be administered to a test animal, preferably a test animal predisposed to develop a type of tumor, and the test animal subsequently examined for an decreased incidence of tumor formation in comparison with controls not administered the compound. Alternatively, a compound can be administered to test animals having tumors (e.g., animals in which tumors have been induced by introduction of malignant, neoplastic, or transformed cells, or by administration of a carcinogen) and subsequently examining the tumors in the test animals for tumor regression in comparison to controls not administered the compound.

One measure of inhibition is IC₅₀, defined as the concentration of the compound at which the activity of KSP is decreased by fifty percent relative to a control. Preferred compounds have IC₅₀'s of less than about 1 mM, with preferred embodiments having IC₅₀'s of less than about 100 μM, with more preferred embodiments having IC₅₀'s of less than about 10 μM, with particularly preferred embodiments having IC₅₀'s of less than about 1 μM, and especially preferred embodiments having IC₅₀'s of less than about 100 nM, and with the most preferred embodiments having IC₅₀'s of less than about 10 nM. Measurement of IC₅₀ is done using an ATPase assay such as described herein.

Another measure of inhibition is Kᵢ. For compounds with IC₅₀'s less than 1 μM, the Kᵢ or K_{d} is defined as the dissociation rate constant for the interaction of the compounds described herein with KSP. Preferred compounds have Kᵢ's of less than about 100 μM, with preferred embodiments having Kᵢ's of less than about 10 μM, and particularly preferred embodiments having Kᵢ's of less than about 1 μM and especially preferred embodiments having Kᵢ's of less than about 100 nM, and with the most preferred embodiments having Kᵢ's of less than about 10 nM.

The Kᵢ for a compound is determined from the IC₅₀ based on three assumptions and the Michaelis-Menten equation. First, only one compound molecule binds to the enzyme and there is no cooperativity. Second, the concentrations of active enzyme and the compound tested are known (i.e., there are no significant amounts of impurities or inactive forms in the preparations). Third, the enzymatic rate of the enzyme-inhibitor complex is zero. The rate (i.e., compound concentration) data are fitted to the equation:

$$V = {V}_{max} {E}_{0} [ I - \frac{( {E}_{0} + {I}_{0} + Kd ) - \sqrt{{( {E}_{0} + {I}_{0} + Kd )}^{2} - 4 {E}_{0} {I}_{0}}}{2 {E}_{0}} ]$$

where V is the observed rate, Vₘₐₓ is the rate of the free enzyme, I₀ is the inhibitor concentration, E₀ is the enzyme concentration, and K_{d} is the dissociation constant of the enzyme-inhibitor complex.

Another measure of inhibition is GI₅₀, defined as the concentration of the compound that results in a decrease in the rate of cell growth by fifty percent. Preferred compounds have GI₅₀'s of less than about 1 mM; those having a GI₅₀ of less than about 20 μM are more preferred; those having a GI₅₀ of less than about 10 μM more so; those having a GI₅₀ of less than about 1 μM more so; those having a GI₅₀ of less than about 100 nM more so; and those having a GI₅₀ of less than about 10 nM even more so. Measurement of GI₅₀ is done using a cell proliferation assay such as described herein. Compounds of this class were found to inhibit cell proliferation.

In vitro potency of small molecule inhibitors is determined, for example, by assaying human ovarian cancer cells (SKOV3) for viability following a 72-hour exposure to a 9-point dilution series of compound. Cell viability is determined by measuring the absorbance of formazon, a product formed by the bioreduction of MTS/PMS, a commercially available reagent. Each point on the dose-response curve is calculated as a percent of untreated control cells at 72 hours minus background absorption (complete cell kill).

Anti-proliferative compounds that have been successfully applied in the clinic to treatment of cancer (cancer chemotherapeutics) have GI50's that vary greatly. For example, in A549 cells, paclitaxel GI₅₀ is 4 nM, doxorubicin is 63 nM, 5-fluorouracil is 1 μM, and hydroxyurea is 500 μM (data provided by National Cancer Institute, Developmental Therapeutic Program, http://dtp.nci.nih.gov/). Therefore, compounds that inhibit cellular proliferation, irrespective of the concentration demonstrating inhibition, have potential clinical usefulness.

Accordingly, the compounds of the invention are administered to cells. By “administered” herein is meant administration of a therapeutically effective amount (dose) of a compound of the invention to a cell either in a cell culture or in a patient. By “therapeutically effective amount” herein is meant an amount that produces the effects for which it is administered. The exact amount will depend on the purpose of the treatment, and will be ascertainable by one skilled in the art using known techniques. As is known in the art, adjustments for systemic versus localized delivery, age, body weight, general health, sex, diet, time of administration, drug interaction and the severity of the condition may be necessary, and will be ascertainable with routine experimentation by those skilled in the art. By “cells” herein is meant any cell in which mitosis or meiosis can be altered.

A “patient” for the purposes of the present invention includes both humans and other animals, particularly mammals, and other organisms. Thus the methods are applicable to both human therapy and veterinary applications. In a particular embodiment the patient is a mammal, and more particularly, the patient is human.

Compounds of the invention having the desired pharmacological activity may be administered, especially as a pharmaceutically acceptable composition comprising an pharmaceutical excipient, to a patient, as described herein. Depending upon the manner of introduction, the compounds may be formulated in a variety of ways as discussed below. The concentration of therapeutically active compound in the formation may vary from about 0.1-10 wt. %.

The agents may be administered alone or in combination with other treatments, i.e., radiation, or other chemotherapeutic agents such as the taxane class of agents that appear to act on microtubule formation or the camptothecin class of topoisomerase I inhibitors. When used, other chemotherapeutic agents may be administered before, concurrently, or after administration of a compound of the present invention. In one aspect of the invention, a compound of the present invention is co-administered with one or more other chemotherapeutic agents. By “co-administer” it is meant that the present compounds are administered to a patient such that the present compounds as well as the co-administered compound may be found in the patient's bloodstream at the same time, regardless when the compounds are actually administered, including simultaneously.

The administration of the compounds and compositions of the present invention can be done in a variety of ways, including, but not limited to, orally, subcutaneously, intravenously, intranasally, transdermally, intraperitoneally, intramuscularly, intrapulmonary, vaginally, rectally, or intraocularly. In some instances, for example, in the treatment of wounds and inflammation, the compound or composition may be directly applied as a solution or spray.

Pharmaceutical dosage forms include a compound of Formula I or a pharmaceutically acceptable salt, solvate, or solvate of a salt thereof, and one or more pharmaceutical excipients. As is known in the art, pharmaceutical excipients are secondary ingredients which function to enable or enhance the delivery of a drug or medicine in a variety of dosage forms (e.g.: oral forms such as tablets, capsules, and liquids; topical forms such as dermal, opthalmic, and otic forms; suppositories; injectables; respiratory forms and the like). Pharmaceutical excipients include inert or inactive ingredients, synergists or chemicals that substantively contribute to the medicinal effects of the active ingredient. For example, pharmaceutical excipients may function to improve flow characteristics, product uniformity, stability, taste, or appearance, to ease handling and administration of dose, for convenience of use, or to control bioavailability. While pharmaceutical excipients are commonly described as being inert or inactive, it is appreciated in the art that there is a relationship between the properties of the pharmaceutical excipients and the dosage forms containing them.

Pharmaceutical excipients suitable for use as carriers or diluents are well known in the art, and may be used in a variety of formulations. See, e.g., Remington's Pharmaceutical Sciences, 18th Edition, A. R. Gennaro, Editor, Mack Publishing Company (1990); Remington: The Science and Practice of Pharmacy, 20th Edition, A. R. Gennaro, Editor, Lippincott Williams & Wilkins (2000); Handbook of Pharmaceutical Excipients, 3rd Edition, A. H. Kibbe, Editor, American Pharmaceutical Association, and Pharmaceutical Press (2000); and Handbook of Pharmaceutical Additives, compiled by Michael and Irene Ash, Gower (1995), each of which is incorporated herein by reference for all purposes.

Oral solid dosage forms such as tablets will typically comprise one or more pharmaceutical excipients, which may for example help impart satisfactory processing and compression characteristics, or provide additional desirable physical characteristics to the tablet. Such pharmaceutical excipients may be selected from diluents, binders, glidants, lubricants, disintegrants, colors, flavors, sweetening agents, polymers, waxes or other solubility-retarding materials.

Compositions for intravenous administration will generally comprise intravenous fluids, i.e., sterile solutions of simple chemicals such as sugars, amino acids or electrolytes, which can be easily carried by the circulatory system and assimilated. Such fluids are prepared with water for injection USP.

Dosage forms for parenteral administration will generally comprise fluids, particularly intravenous fluids, i.e., sterile solutions of simple chemicals such as sugars, amino acids or electrolytes, which can be easily carried by the circulatory system and assimilated. Such fluids are typically prepared with water for injection USP. Fluids used commonly for intravenous (IV) use are disclosed in Remington, The Science and Practice of Pharmacy [full citation previously provided], and include:
alcohol, e.g., 5% alcohol (e.g., in dextrose and water (“D/W”) or D/W in normal saline solution (“NSS”), including in 5% dextrose and water (“D5/W”), or D5/W in NSS);
synthetic amino acid such as Aminosyn, FreAmine, Travasol, e.g., 3.5 or 7; 8.5; 3.5, 5.5 or 8.5% respectively;
ammonium chloride e.g., 2.14%;
dextran 40, in NSS e.g., 10% or in D5/W e.g., 10%;
dextran 70, in NSS e.g., 6% or in D5/W e.g., 6%;
dextrose (glucose, D5/W) e.g., 2.5-50%;
dextrose and sodium chloride e.g., 5-20% dextrose and 0.22-0.9% NaCl;
lactated Ringer's (Hartmann's) e.g., NaCl 0.6%, KCl 0.03%, CaCl₂ 0.02%;
lactate 0.3%;
mannitol, e.g., 5%, optionally in combination with dextrose e.g., 10% or NaCl e.g., 15 or 20%;
multiple electrolyte solutions with varying combinations of electrolytes, dextrose, fructose, invert sugar Ringer's e.g., NaCl 0.86%, KCl 0.03%, CaCl₂ 0.033%;
sodium bicarbonate e.g., 5%;
sodium chloride e.g., 0.45, 0.9, 3, or 5%;
sodium lactate e.g., 1/6 M; and
sterile water for injection
   The pH of such fluids may vary, and will typically be from 3.5 to 8 as known in the art.

The compounds, pharmaceutically acceptable salts and solvates of the invention can be administered alone or in combination with other treatments, i.e., radiation, or other therapeutic agents, such as the taxane class of agents that appear to act on microtubule formation or the camptothecin class of topoisomerase I inhibitors. When so-used, other therapeutic agents can be administered before, concurrently (whether in separate dosage forms or in a combined dosage form), or after administration of an active agent of the present invention.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All publications, including but not limited to patents and patent applications, cited in this specification are herein incorporated by reference as if each individual publication were specifically and individually indicated to be incorporated by reference herein as though fully set forth.

### EXAMPLE 1

### General Synthesis of dihydropyrimidin-2 (1H)-ones (3)

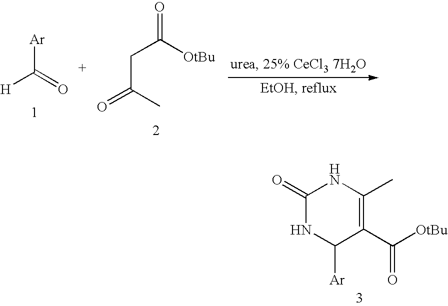

1.0 mmol of aldehyde, 160 μL (1.0 mmol) of ethyl acetoacetate, 180 mg (3.0 mmol) of urea, and 94 mg (0.25 mmol) of CeCl₃.7H₂O are combined in 3 mL of EtOH and heated at 80° C. for 4 hours. The reactions are allowed to cool and left for one day. If solid formed, it is filtered and rinsed with cold ethanol. If no solid formed the reaction mixture is poured into cold water and stirred for 10 minutes. The resulting solid is then filtered and rinsed with cold ethanol. Further purification by flash chromatography or reverse-phase HPLC may be performed.

Using the method described above and the aldehydes shown in the table below, the corresponding products shown in the table below were prepared.

| Entry | Aldehyde (1) | Product | Yield % |
|---|---|---|---|
| 1 | 3-Chloro-4-hydroxy-5-methoxy-benzaldehyde | 3.1 | 62 |
| 2 | 4-Hydroxy-3-iodo-5-methoxy-benzaldehyde | 3.2 | 66 |
| 3 | 4-Hydroxy-3-methoxy-benzaldehyde | 3.3 | 55 |
| 4 | 3-Bromo-4-hydroxy-benzaldehyde | 3.4 | 33 |
| 5 | 4-Hydroxy-3,5-dimethoxy-benzaldehyde | 3.5 | 16 |
| 6 | 3,5-Dichloro-4-hydroxy-benzaldehyde | 3.6 | 31 |
| 7 | 3-Bromo-5-ethoxy-4-hydroxy-benzaldehyde | 3.7 | 48 |
| 8 | 3-Chloro-4,5-dimethoxy-benzaldehyde | 3.8 | 20 |
| 9 | 3-Fluoro-4-hydroxy-5-methoxy-benzaldehyde | 3.9 | 50 |
| 10 | 4-Hydroxy-3-methoxy-5-nitro-benzaldehyde | 3.10 | 76 |
| 11 | 4-Fluoro-3-methoxy-benzaldehyde | 3.11 | 38 |
| 12 | 1H-Indole-5-carbaldehyde | 3.12 | 15 |
| 13 | 3H-Imidazole-4-carbaldehyde | 3.13 | 32 |

### EXAMPLE 2

### General Synthesis of amide Substituted dihydropyrimidin-2 (1H)-ones (5):

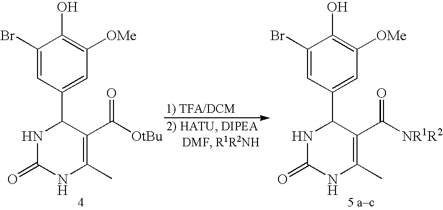

| | R¹ | R² | yield (%) |
|---|---|---|---|
| 5a | H | Me | 29 |
| 5b | Me | Me | 17 |
| 5c | H | tBu | 38 |

Compound 4 was synthesized using the general procedure for dihydropyrimidin-2 (1H)-ones detailed above. 2.00 g (4.85 mmol) of the crude ester was dissolved in 25 mL of methylene chloride and 25 mL of trifluoracetic acid. The mixture was left for 0.5 hour and then concentrated on a rotary evaporator to give an oily residue which was then evaporated four times from toluene to yield a tan solid. The crude acid was washed with diethyl ether, dried under vacuum, and used without further purification (1.65 g, 96%). 400 mg (1.12 mmol) of 4 was combined with 425 mg (1.12 mml) of O-(7-Azabenzotriazol-1-yl)-N,N,N′,N′-tetramethyluronium hexafluorophosphate (HATU) in 2.0 mL of sieve-dried dimethylormamide. 80 uL (4.11 mmol) of N,N-diisopropylethylamine was added followed immediately by 10-15 equivalents of the appropriate amine in a solution of tetrahydrofuran. After stirring for 14 to 16 hours the solution was concentrated on a rotary evaporator. The residue was then dissolved in 25 mL of methanol, approximately 5 mL of silica gel was added, and the mixture evaporated to dryness. The silica was added to a short pad of methylene chloride/silica and the product eluted with 10% methanol/methylene chloride. The product was then washed with methanol or acetonitrile and dried under vacuum.

### EXAMPLE 3

### Synthesis of aniline and acetamide Substituted dihydropyrimidin-2 (1H)-ones (7-8)

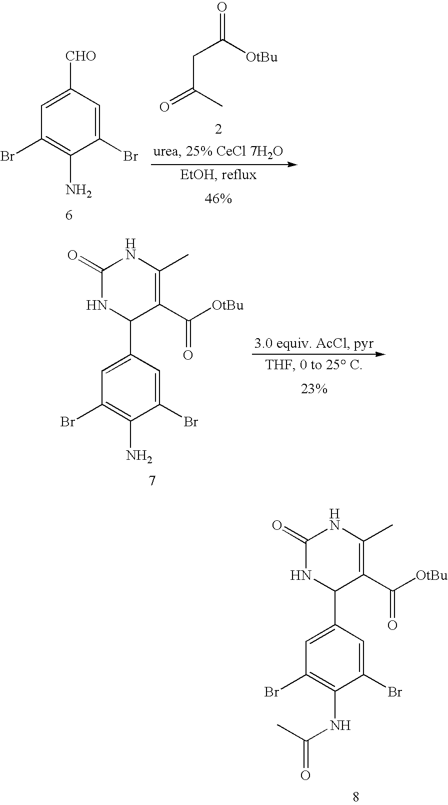

140 mg (0.5 mmol) of 4-amino-3,5-dibromo-benzaldehyde, 80 μL 0.5 mmol of ethyl acetoacetate, 180 mg (3.0 mmol) of urea, and 94 mg (0.25 mmol) of CeCl₃.7H₂O were combined in 3 mL of EtOH and heated at reflux for 14 hours. The reaction was cooled, filtered, and washed with cold ethanol. The tan solid was dried under vacuum to provide 107 mg of 7 (46%).

40 mg (0.09 mmol) of 7 was dissolved in 1 mL dry tetrahydrofuran with 21 μL (0.26 mmol) of pyridine and cooled in an ice-bath. 20 μL (0.20 mmol) of acetyl chloride was dissolved in 0.2 mL of tetrahydrofuran and added to the above solution in small portions over 1 hour. The reaction was then allowed to warm to r.t. and concentrated on a rotary evaporator. The residue was purified by preparative silica TLC (5% methanol/methylene choride) to yield 26 mg (23%) of 8 as a white solid.

### EXAMPLE 4

### Synthesis of N1 Substituted dihydropyrimidin-2(1H)-ones (11a-b):

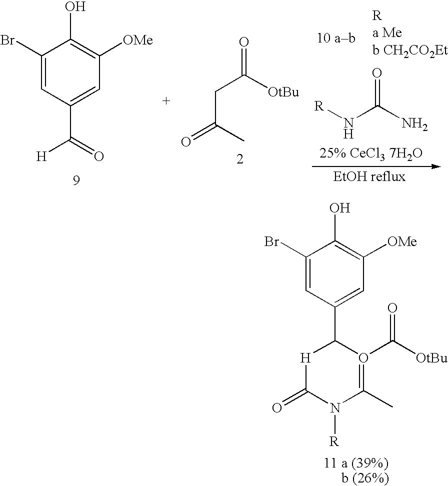

Compounds 11a-b were synthesized using the general procedure for the synthesis of dihydropyrimidin-2 (1H)-ones. The reactions were heated at reflux for 15 hours, cooled, filtered, and washed with ethanol.

### EXAMPLE 5

### Synthesis of N1 benzyl Substituted dihydropyrimidin-2 (1H)-one (14)

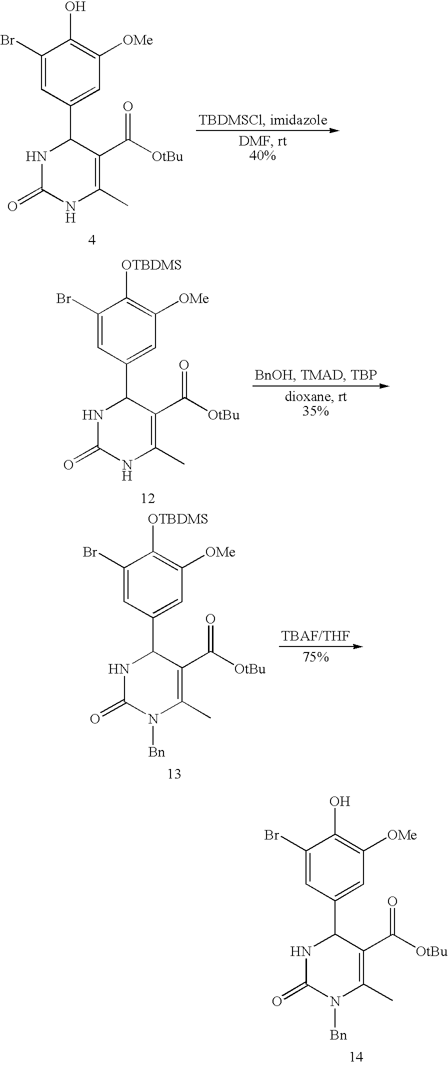

1.14 g (2.76 mmol) of 4 was combined with 0.19 g (2.80 mmol) of imidazole and 0.42 g (2.80 mmol) of tert-butyidimethylsilyl chloride in 20 mL of sieve-dried dimethylformamide and stirred at r.t. for 18 hours. The reaction was diluted with 100 mL of ethyl acetate and washed 4 times with 100 mL of saturated brine solution. The ethyl acetate solution was dried with Na₂SO₄ and concentrated under reduced pressure to give a yellow oil. Flash chromatography (1% methanol/methylene chloride) of the oil followed by removal of solvent yielded 578 mg (40%) of 12 as off-white foam.

420 mg (0.80 mmol) of 12 was dissolved in 5 mL sieve-dried dioxane. 0.42 mL (4.0 mmol) of benzyl alcohol was added followed by 0.50 mL (2.0 mmol) tributylphosphine and 344 mg (2.0 mmol) of 1,1′-azo-bis (N,N-dimethylformamide). The reaction mixture was purged with nitrogen for 10 minutes, sealed and stirred at r.t. for 19 hours. It was then filtered, rinsed with tetrahydrofuran and concentrated under reduce pressure. Flash chromatography (1-4% methanol/methylene chloride) of the oil followed by removal of solvent yielded 185 mg (35%) of 13 as a clear, colorless film.

185 mg (0.30 mmol) of 13 was dissolved in 5 mL of tetrahydrofuran and 0.60 mL (0.60 mmol) of 1M tetrabutylammonium fluoride/tetrahydrofuran solution was added. The reaction was left at r.t. for 20 hours when approximately 50 mL of saturated aqueous ammonium chloride was added. After 2 days the white precipitate that had formed was filtered, rinsed with diethyl ether and dried under vacuum to give 110 mg (75%) of 14 as a white solid.

### EXAMPLE 6

### Synthesis of N1, N3 Di-Substituted dihydropyrimidin-2 (1H-one (16)

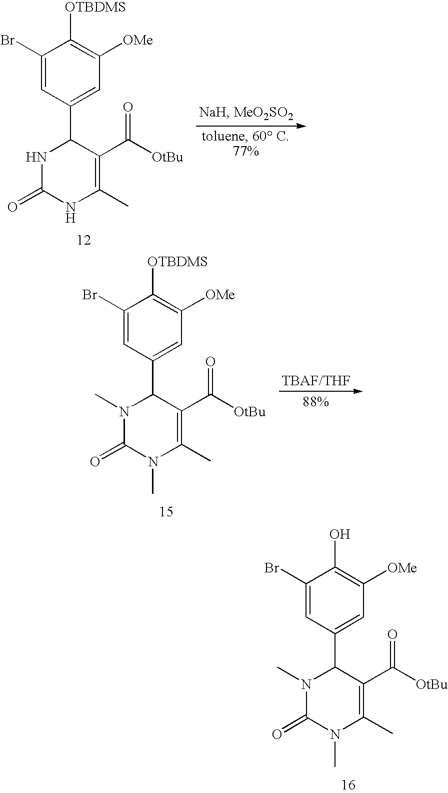

35 mg (0.07 mmol) of 12 was dissolved in 2 mL of toluene with 25 mg (0.20 mmol) dimethyl sulfate. 14 mg (0.35 mmol) of sodium hydride (60% suspension in mineral oil) was added in small portions and the reaction sealed. After heating at 60° C. for 0.5 hour, 2 mL of saturated aqueous ammonium chloride was added along with 3 mL of ethyl acetate. The organic layer was washed with saturated brine and concentrated to provide 30 mg (77%) of 15 as a clear, colorless film.

30 mg (0.054 mmol) of 15 was dissolved in 1 mL tetrahydrofuran and 0.16 mL (0.16 mmol) 1M tetrabutylammonium fluoride/tetrahydrofuran solution was added. After 1 hour, 5 mL of saturated aqueous ammonium chloride was added along with 10 mL ethyl acetate. The organic phase was washed with brine, dried with Na₂SO₄ and concentrated under reduded pressure. The residue was purified using preparative silica TLC (5% methanol/methylene chloride) to yield 21 mg (88%) of 16 as a white powder.

### EXAMPLE 7

### Inhibition of Cellular Proliferation in Tumor Cell Lines

Cells are plated in 96-well plates at densities from 1000-2500 cells/well of a 96-well plate and allowed to adhere/grow for 24 hours. They are then treated with various concentrations of a compound of the invention for 48 hours. The time at which compounds are added is considered T₀. A tetrazolium-based assay using the reagent 3-(4,5-dimethylthiazol-2-yl)-5-(3-carboxymethoxyphenyl)-2-(4-sulfophenyl)-2H-tetrazolium (MTS) (U.S. Pat. No. 5,185,450) (see Promega product catalog #G3580, CellTiter 96® AQᵤₑₒᵤₛ One Solution Cell Proliferation Assay) is used to determine the number of viable cells at T₀ and the number of cells remaining after 48 hours compound exposure. The number of cells remaining after 48 hours is compared to the number of viable cells at the time of compound addition, allowing for calculation of growth inhibition.

The growth over 48 hours of cells in control wells that have been treated with vehicle only (0.25% DMSO) is considered 100% growth and the growth of cells in wells with compounds is compared to this.

A GI₅₀ is calculated by plotting the concentration of compound in μM vs the percentage of cell growth in treated wells. The Gi₅₀ calculated for the compounds is the estimated concentration at which growth is inhibited by 50% compared to control, i.e., the concentration at which:
100×[(Treated₄₈−T₀)/ (Control₄₈−T₀)]=50
wherein Treated₄₈ is the value at 48 hours for the treated cells and Control₄₈ is the value at 48 hours for the control population.

All concentrations of compounds are tested in duplicate and controls are averaged over 12 wells. A very similar 96-well plate layout and Gi₅₀ calculation scheme is used by the National Cancer Institute (see Monks, et al., J. Natl. Cancer Inst. 83:757-766 (1991)). However, the method by which the National Cancer Institute quantitates cell number does not use MTS, but instead employs alternative methods.

1. A compound of Formula I 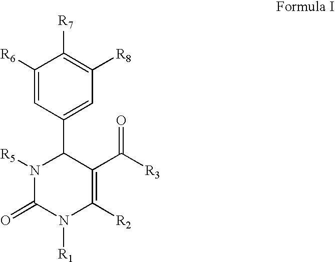
or a pharmaceutically acceptable salt, solvate, crystal form, diastereomer, or prodrug thereof, wherein
R₁ and R₅ are each independently hydrogen, optionally substituted lower alkyl, optionally substituted aryl or optionally substituted heteroaryl;
R₂ is hydrogen, optionally substituted lower alkyl, optionally substituted aryl or optionally substituted heteroaryl;
R₃ is optionally substituted alkoxy, optionally substituted alkyl, or NR₉R₁₀ wherein R₉ and R₁₀ are independently selected from hydrogen, optionally substituted lower alkyl, optionally substituted aryl or optionally substituted heteroaryl;
R₆ is hydrogen, cyano, nitro, halo, optionally substituted alkyl, or optionally substituted alkoxy;
R₇ is halo, optionally substituted alkyl, cyano, nitro, hydroxy, optionally substituted alkoxy, or optionally substituted amino; and
R₈ is halo, optionally substituted alkyl, or optionally substituted alkoxy;
or R₆ and R₇, together with the carbons to which they are attached, form an optionally substituted 3- to 7-membered ring which optionally includes one or more heteroatoms selected from N, O, and S in the ring; and R₈ is halo, optionally substituted alkyl, or optionally substituted alkoxy;
or R₇ and R₈, together with the carbons to which they are attached, form an optionally substituted 3- to 7-membered ring which optionally includes one or more heteroatoms selected from N, O, and S in the ring; and R₆ is hydrogen, cyano, nitro, halo, optionally substituted alkyl, or optionally substituted alkoxy;
provided that only one of R₆, R₇, and R₈ is optionally substituted alkoxy; and
provided that when R₁ and R₅ are hydrogen, R₂ is methyl, R₃ is methoxy or ethoxy, R₇ is hydroxy, and R₈ is methoxy, then R₆ is not hydrogen or halo.

2. A compound according to claim 1 wherein R₁ and R₅ are independently hydrogen or optionally substituted lower alkyl.

3. A compound according to claim 2 wherein R₁ is hydrogen, methyl, ethyl, benzyl, 2-(N,N-dimethylamino)ethyl, carboxymethyl, (ethyoxy)carbonylmethyl, or (2-methoxyethylcarbamoyl)methyl.

4. A compound according to claim 3 wherein R₁ is hydrogen, methyl, or ethyl.

5. A compound according to claim 4 wherein R₁ is hydrogen.

6. A compound according to claim 1 wherein R₁ and R₅ are hydrogen.

7. A compound according to claim 1 wherein R₂ is optionally substituted lower alkyl.

8. A compound according to claim 7 wherein R₂ is optionally substituted methyl.

9. A compound according to claim 8 wherein R₂ is methyl, halomethyl, alkoxycarbonylmethyl-, carboxymethyl-, alkoxymethyl, or hydroxymethyl.

10. A compound according to claim 9 wherein R₂ is methyl.

11. A compound according to claim 1 wherein R₃ is optionally substituted alkoxy.

12. A compound according to claim 11 wherein R₃ is optionally substituted C₁-C₈ alkoxy.

13. A compound according to claim 12 wherein R₃ is methoxy-, ethoxy-, propoxy-, isopropoxy-, butoxy-, pentoxy-, c-pentoxy-, hexoxy-, c-hexyloxy-, heptoxy, c-heptoxy-, 1,2,2-trimethylpropoxy-, 1,1,1,-trifluoro-isopropoxy-, 1-methyl-propoxy-, 2-methyl-propoxy-, 3-methyl-butoxy-, t-butoxy-, benzyloxy-, 4-methyl-benzyloxy-, or 1-(2-isopropoxyethoxy).

14. A compound according to claim 1 wherein R₆ is hydrogen, bromo, chloro, fluoro, methyl, trifluoromethyl, hydroxymethyl, or methoxy.

15. A compound according to claim 1 wherein R₇ is hydroxy, optionally substituted alkoxy, or optionally substituted amino.

16. A compound according to claim 15 wherein R₇ is hydroxy.

17. A compound according to claim 15 wherein R₇ is optionally substituted lower alkoxy.

18. A compound according to claim 17 wherein R₇ is methoxy.

19. A compound according to claim 15 15 wherein R₇ is acetylamino or amino.

20. A compound according to claim 1 wherein R₆ and R₇, together with the carbons to which they are attached, form an optionally substituted 3- to 7-membered ring which optionally includes one or more heteroatoms selected from N, O, and S in the ring.

21. A compound according to claim 20 wherein R₆ and R₇, together with the phenyl ring to which they are attached, form a substituted 1H-indole, 2,3-dihydro-benzo[1,4]dioxine, substituted benzimidazole, or substituted benzo[1,3]dioxole.

22. A compound according to claim 1 wherein R₇ and R₈, together with the carbons to which they are attached, form an optionally substituted 3- to 7-membered ring which optionally includes one or more heteroatoms selected from N, O, and S in the ring.

23. A compound according to claim 1 wherein R₈ is bromo, chloro, fluoro, methyl, trifluoromethyl, hydroxymethyl, or methoxy.

24. A compound according to claim 1 wherein at least one of R₆ and R₈ is halo.

25. A compound according to claim 1 wherein at least one of R₆ and R₈ is optionally substituted alkoxy.

26. A compound according to claim 1 wherein at least one of R₆ and R₈ is trifluoromethyl.

27. A compound according to claim 1 wherein at least one of R₆ and R₈ is methyl.

28. A compound according to claim 1 wherein
R₁ and R₅ are independently hydrogen or optionally substituted lower alkyl;
R₂ is optionally substituted lower alkyl;
R₃ is optionally substituted alkoxy or optionally substituted alkyl;
R₆ is hydrogen, cyano, nitro, halo, optionally substituted alkyl, or optionally substituted alkoxy;
R₇ is halo, optionally substituted alkyl, cyano, nitro, hydroxy, optionally substituted alkoxy, or optionally substituted amino; and
R₈ is halo, optionally substituted alkyl, or optionally substituted alkoxy.

29. A compound according to claim 1 wherein
R₁ and R₅ are independently hydrogen or optionally substituted lower alkyl;
R₂ is optionally substituted lower alkyl;
R₃ is optionally substituted alkoxy or optionally substituted alkyl;
R₆ is hydrogen, bromo, chloro, fluoro, methyl, trifluoromethyl, or methoxy;
R₇ is halo, optionally substituted alkyl, cyano, nitro, hydroxy, optionally substituted alkoxy, or optionally substituted amino; and
R₈ is halo, optionally substituted alkyl, or optionally substituted alkoxy.

30. A compound according to claim 1 wherein
R₁ and R₅ are independently hydrogen or optionally substituted lower alkyl;
R₂ is optionally substituted lower alkyl;
R₃ is optionally substituted alkoxy or optionally substituted alkyl;
R₆ is hydrogen, cyano, nitro, halo, optionally substituted alkyl, or optionally substituted alkoxy;
R₇ is hydroxy; and
R₈ is halo, optionally substituted alkyl, or optionally substituted alkoxy.

31. A compound according to claim 1 wherein
R₁ and R₅ are independently hydrogen or optionally substituted lower alkyl;
R₂ is optionally substituted lower alkyl;
R₃ is optionally substituted alkoxy or optionally substituted alkyl;
R₆ is hydrogen, cyano, nitro, halo, optionally substituted alkyl, or optionally substituted alkoxy;
R₇ is methoxy; and
R₈ is halo, optionally substituted alkyl, or optionally substituted alkoxy.

32. A compound according to claim 1 wherein
R₁ and R₅ are independently hydrogen or optionally substituted lower alkyl;
R₂ is optionally substituted lower alkyl;
R₃ is optionally substituted alkoxy or optionally substituted alkyl;
R₆ is hydrogen, cyano, nitro, halo, optionally substituted alkyl, or optionally substituted alkoxy;
R₇ is amino; and
R₈ is halo, optionally substituted alkyl, or optionally substituted alkoxy.

33. A compound according to claim 1 wherein
R₁ and R₅ are independently hydrogen or optionally substituted lower alkyl;
R₂ is optionally substituted lower alkyl;
R₃ is optionally substituted alkoxy or optionally substituted alkyl;
R₆ is hydrogen, cyano, nitro, halo, optionally substituted alkyl, or optionally substituted alkoxy;
R₇ is halo, optionally substituted alkyl, cyano, nitro, hydroxy, optionally substituted alkoxy, or optionally substituted amino; and
R₈ is bromo, chloro, fluoro, methyl, trifluoromethyl, or methoxy.

34. A compound according to claim 1 wherein
R₁ and R₅ are independently hydrogen or optionally substituted lower alkyl;
R₂ is optionally substituted lower alkyl;
R₃ is optionally substituted alkoxy or optionally substituted alkyl;
R₆ is hydrogen, bromo, chloro, fluoro, methyl, trifluoromethyl, or methoxy;
R₇ is hydroxy; and
R₈ is bromo, chloro, fluoro, methyl, trifluoromethyl, or methoxy.

35. A compound according to claim 1 wherein
R₁ and R₅ are independently hydrogen or optionally substituted lower alkyl;
R₂ is optionally substituted lower alkyl;
R₃ is optionally substituted alkoxy or optionally substituted alkyl;
R₆ is hydrogen, bromo, chloro, fluoro, methyl, trifluoromethyl, or methoxy;
R₇ is methoxy; and
R₈ is bromo, chloro, fluoro, methyl, trifluoromethyl, or methoxy.

36. A compound according to claim 1 wherein
R₁ and R₅ are independently hydrogen or optionally substituted lower alkyl;
R₂ is optionally substituted lower alkyl;
R₃ is optionally substituted alkoxy or optionally substituted alkyl;
R₆ is hydrogen, bromo, chloro, fluoro, methyl, trifluoromethyl, or methoxy;
R₇ is amino; and
R₈ is bromo, chloro, fluoro, methyl, trifluoromethyl, or methoxy.

37. A compound according to claim 1 wherein
R₁ and R₅ are independently hydrogen or optionally substituted lower alkyl;
R₂ is optionally substituted lower alkyl;
R₃ is optionally substituted alkoxy or optionally substituted alkyl;
R₆ is hydrogen, cyano, nitro, halo, optionally substituted alkyl, or optionally substituted alkoxy;
R₇ is halo, optionally substituted alkyl, cyano, nitro, hydroxy, optionally substituted alkoxy, or optionally substituted amino; and
R₈ is halo, optionally substituted alkyl, or optionally substituted alkoxy, provided that at least one of R₆ and R₈ is halo.

38. A compound according to claim 1 wherein
R₁ and R₅ are independently hydrogen or optionally substituted lower alkyl;
R₂ is optionally substituted lower alkyl;
R₃ is optionally substituted alkoxy or optionally substituted alkyl;
R₆ is hydrogen, cyano, nitro, halo, optionally substituted alkyl, or optionally substituted alkoxy;
R₇ is halo, optionally substituted alkyl, cyano, nitro, hydroxy, optionally substituted alkoxy, or optionally substituted amino; and
R₈ is halo, optionally substituted alkyl, or optionally substituted alkoxy; provided that at least one of R₆ and R₈ is optionally substituted alkoxy.

39. A compound according to claim 1 that is
3,3-dimethylbutan-2-yl 4-(3-bromo-4-hydroxy-5-methoxyphenyl)-1,2,3,4-tetrahydro-methyl-2-oxopyrimidine-5-carboxylate;
cyclopentyl 4-(3-bromo-4-hydroxy-5-methoxyphenyl)-1,2,3,4-tetrahydro-1,6-dimethyl-2-oxopyrimidine-5-carboxylate;
3,3-dimethylbutan-2-yl 4-(3-chloro-4-hydroxy-5-methoxyphenyl)-1,2,3,4-tetrahydro-6-methyl-2-oxopyrimidine-5-carboxylate;
sec-butyl 4-(3-bromo-4-hydroxy-5-methoxyphenyl)-1,2,3,4-tetrahydro-6-methyl-2-oxopyrimidine-5-carboxylate;
isopentyl 4-(3-bromo-4-hydroxy-5-methoxyphenyl)-1,2,3,4-tetrahydro-1,6-dimethyl-2-oxopyrimidine-5-carboxylate;
ethyl 4-(3-bromo-4-methoxyphenyl)-1,2,3,4-tetrahydro-6-methyl-2-oxopyrimidine-5-carboxylate;
isobutyl 4-(3-chloro-4-hydroxy-5-methoxyphenyl)-1,2,3,4-tetrahydro-6-methyl-2-oxopyrimidine-5-carboxylate;
cyclopentyl 4-(3-chloro-4-hydroxy-5-methoxyphenyl)-1,2,3,4-tetrahydro-1,6-dimethyl-2-oxopyrimidine-5-carboxylate;
sec-butyl 4-(3-chloro-4-hydroxy-5-methoxyphenyl)-1,2,3,4-tetrahydro-6-methyl-2-oxopyrimidine-5-carboxylate;
ethyl 4-(benzo[d][1,3]dioxol-5-yl)-6-(chloromethyl)-1,2,3,4-tetrahydro-2-oxopyrimidine-5-carboxylate;
3,3-dimethylbutan-2-yl 1,2,3,4-tetrahydro-4-(4-hydroxy-3-methoxy-5-methylphenyl)-6-methyl-2-oxopyrimidine-5-carboxylate;
3,3-dimethylbutan-2-yl 1-((ethoxycarbonyl)methyl)-4-(3-bromo-4-hydroxy-5-methoxyphenyl)-1,2,3,4-tetrahydro-6-methyl-2-oxopyrimidine-5-carboxylate;
3,3-dimethylbutan-2-yl 4-(3-bromo-4-hydroxy-5-methoxyphenyl)-1-ethyl-1,2,3,4-tetrahydro-6-methyl-2-oxopyrimidine-5-carboxylate;
cyclopentyl 4-(3-chloro-4-hydroxy-5-methoxyphenyl)-1,2,3,4-tetrahydro-1,6-dimethyl-2-oxopyrimidine-5-carboxylate;
tert-butyl 4-(3-chloro-4-hydroxy-5-methoxyphenyl)-1,2,3,4-tetrahydro-6-methyl-2-oxopyrimidine-5-carboxylate;
cyclohexyl 4-(3-bromo-4-hydroxy-5-methoxyphenyl)-1,2,3,4-tetrahydro-6-methyl-2-oxopyrimidine-5-carboxylate;
sec-butyl 4-(3-chloro-4-hydroxy-5-methoxyphenyl)-1,2,3,4-tetrahydro-6-methyl-2-oxopyrimidine-5-carboxylate;
tert-butyl 1,2,3,4-tetrahydro-4-(4-hydroxy-3-methoxyphenyl)-6-methyl-2-oxopyrimidine-5-carboxylate;
4,4-dimethylpentan-2-yl 1-((2-methoxyethylcarbamoyl)methyl)-4-(3-bromo-4-hydroxy-5-methoxyphenyl)-1,2,3,4-tetrahydro-6-methyl-2-oxopyrimidine-5-carboxylate;
tert-butyl 1-((ethoxycarbonyl)methyl)-4-(3-bromo-4-hydroxy-5-methoxyphenyl)-1,2,3,4-tetrahydro-6-methyl-2-oxopyrimidine-5-carboxylate;
tert-butyl 1-benzyl-4-(3-bromo-4-hydroxy-5-methoxyphenyl)-1,2,3,4-tetrahydro-6-methyl-2-oxopyrimidine-5-carboxylate;
cycloheptyl 4-(3-chloro-4-hydroxy-5-methoxyphenyl)-1,2,3,4-tetrahydro-6-methyl-2-oxopyrimidine-5-carboxylate;
tert-butyl 1,2,3,4-tetrahydro-4-(1H-indol-5-yl)-6-methyl-2-oxopyrimidine-5-carboxylate;
sec-butyl 4-(3-bromo-4-hydroxy-5-methoxyphenyl)-1,2,3,4-tetrahydro-6-methyl-2-oxopyrimidine-5-carboxylate;
3,3-dimethylbutan-2-yl 4-(3-fluoro-4-hydroxy-5-methoxyphenyl)-1,2,3,4-tetrahydro-6-methyl-2-oxopyrimidine-5-carboxylate;
tert-butyl 1,2,3,4-tetrahydro-4-(4-hydroxy-3-methoxy-5-methylphenyl)-6-methyl-2-oxopyrimidine-5-carboxylate;
tert-butyl 4-(3-bromo-5-ethoxy-4-hydroxyphenyl)-1,2,3,4-tetrahydro-6-methyl-2-oxopyrimidine-5-carboxylate;
ethyl 4-(3-bromo-4-methoxyphenyl)-1,2,3,4-tetrahydro-6-methyl-2-oxopyrimidine-5-carboxylate;
tert-butyl 4-(3-bromo-4-hydroxy-5-methoxyphenyl)-1,2,3,4-tetrahydro-1,6-dimethyl-2-oxopyrimidine-5-carboxylate;
3,3-dimethylbutan-2-yl 1,2,3,4-tetrahydro-4-(4-hydroxy-3-methoxyphenyl)-6-methyl-2-oxopyrimidine-5-carboxylate;
isopropyl 4-(3-chloro-4-hydroxy-5-methoxyphenyl)-1,2,3,4-tetrahydro-6-methyl-2-oxopyrimidine-5-carboxylate;
tert-butyl 4-(3-bromo-4-hydroxy-5-methoxyphenyl)-1,2,3,4-tetrahydro-6-methyl-2-oxopyrimidine-5-carboxylate;
tert-butyl 4-(3-fluoro-4-hydroxy-5-methoxyphenyl)-1,2,3,4-tetrahydro-6-methyl-2-oxopyrimidine-5-carboxylate;
tert-butyl 4-(3-bromo-4-hydroxyphenyl)-1,2,3,4-tetrahydro-6-methyl-2-oxopyrimidine-5-carboxylate
4-(3-bromo-4-hydroxy-5-methoxy-phenyl)-6-methyl-2-oxo-1,2,3,4-tetrahydro-pyrimidine-5-carboxylic acid 1,2,2-trimethyl-propyl ester;
4-(3-Bromo-4-hydroxy-5-methoxy-phenyl)-6-methyl-2-oxo-1,2,3,4-tetrahydro-pyrimidine-5-carboxylic acid isopropyl ester;
4-(3-Bromo-4-hydroxy-5-methoxy-phenyl)-1-carboxymethyl-6-methyl-2-oxo-1,2,3,4-tetrahydro-pyrimidine-5-carboxylic acid 1,2,2-trimethyl-propyl ester;
4-(4-Hydroxy-3-methoxy-5-methyl-phenyl)-6-methyl-2-oxo-1,2,3,4-tetrahydro-pyrimidine-5-carboxylic acid 1,2,2-trimethyl-propyl ester;
4-(2-Bromo-phenyl)-6-methyl-2-oxo-1,2,3,4-tetrahydro-pyrimidine-5-carboxylic acid sec-butyl ester;
4-(3,5-Dichloro-4-hydroxy-phenyl)-6-methyl-2-oxo-1,2,3,4-tetrahydro-pyrimidine-5-carboxylic acid tert-butyl ester;
4-(3,5-Dimethoxy-4-hydroxy-phenyl)-6-methyl-2-oxo-1,2,3,4-tetrahydro-pyrimidine-5-carboxylic acid tert-butyl ester;
4-(4-Hydroxy-3-methoxy-5-nitro-phenyl)-6-methyl-2-oxo-1,2,3,4-tetrahydro-pyrimidine-5-carboxylic acid tert-butyl ester;
4-(4-Fluoro-3-methoxy-phenyl)-6-methyl-2-oxo-1,2,3,4-tetrahydro-pyrimidine-5-carboxylic acid tert-butyl ester;
4-(4-Acetylamino-3,5-dibromo-phenyl)-6-methyl-2-oxo-1,2,3,4-tetrahydro-pyrimidine-5-carboxylic acid tert-butyl ester;
4-(3-Bromo-4-hydroxy-5-methoxy-phenyl)-1,3,6-trimethyl-2-oxo-1,2,3,4-tetrahydro-pyrimidine-5-carboxylic acid tert-butyl ester;
4-(4-Hydroxy-3-methoxy-5-cyano-phenyl)-6-methyl-2-oxo-1,2,3,4-tetrahydro-pyrimidine-5-carboxylic acid 1,2,2-trimethyl-propyl ester;
4-(4-Hydroxy-3-methoxy-5-(hydroxymethyl)-phenyl)-6-methyl-2-oxo-1,2,3,4-tetrahydro-pyrimidine-5-carboxylic acid 1,2,2-trimethyl-propyl ester;
4-(4-Hydroxy-3,5-dimethyl-phenyl)-6-methyl-2-oxo-1,2,3,4-tetrahydro-pyrimidine-5-carboxylic acid 1,2,2-trimethyl-propyl ester;
4-(1H-Benzoimidazol-5-yl)-6-methyl-2-oxo-1,2,3,4-tetrahydro-pyrimidine-5-carboxylic acid 1,2,2-trimethyl-propyl ester;
6-Methyl-4-(7-methyl-1H-benzoimidazol-5-yl)-2-oxo-1,2,3,4-tetrahydro-pyrimidine-5-carboxylic acid 1,2,2-trimethyl-propyl ester;
6-Methyl-4-(7-methoxy-1H-indol-5-yl)-2-oxo-1,2,3,4-tetrahydro-pyrimidine-5-carboxylic acid 1,2,2-trimethyl-propyl ester;
6-Methyl-4-(7-methyl-1H-indol-5-yl)-2-oxo-1,2,3,4-tetrahydro-pyrimidine-5-carboxylic acid 1,2,2-trimethyl-propyl ester;
4-(4-Benzyloxy-3-methoxy-5-methyl-phenyl)-6-methyl-2-oxo-1,2,3,4-tetrahydro-pyrimidine-5-carboxylic acid 1,2,2-trimethyl-propyl ester;
1-Ethyl-4-(4-hydroxy-3-methoxy-5-methyl-phenyl)-6-methyl-2-oxo-1,2,3,4-tetrahydro-pyrimidine-5-carboxylic acid 1,2,2-trimethyl-propyl ester;
1-(2-Dimethylamino-ethyl)-4-(4-hydroxy-3-methoxy-5-methyl-phenyl)-6-methyl-2-oxo-1,2,3,4-tetrahydro-pyrimidine-5-carboxylic acid 1,2,2-trimethyl-propyl ester;
1-Ethoxycarbonylmethyl-4-(4-hydroxy-3-methoxy-5-methyl-phenyl)-6-methyl-2-oxo-1,2,3,4-tetrahydro-pyrimidine-5-carboxylic acid 1,2,2-trimethyl-propyl ester;
4-(3-Bromo-4-hydroxy-5-methoxy-phenyl)-1-[(2-methoxy-ethylcarbamoyl)-methyl]-6-methyl-2-oxo-1,2,3,4-tetrahydro-pyrimidine-5-carboxylic acid 1,2,2-trimethyl-propyl ester;
4-(3-Cyano-4-hydroxy-5-methoxy-phenyl)-6-methyl-2-oxo-1,2,3,4-tetrahydro-pyrimidine-5-carboxylic acid 1,2,2-trimethyl-propyl ester;
4-(3-Bromo-4-hydroxy-5-methoxy-phenyl)-6-methyl-2-oxo-1,2,3,4-tetrahydro-pyrimidine-5-carboxylic acid 1,2,2-trimethyl-propyl ester or
4-(3-Bromo-4-hydroxy-5-methoxy-phenyl)-6-methyl-2-oxo-1,2,3,4-tetrahydro-pyrimidine-5-carboxylic acid cyclopentyl ester.

40. A pharmaceutical composition comprising a therapeutically effective amount of a compound of claim 1 and one or more pharmaceutical excipients.

41. A method of treating a cellular proliferative disease comprising administering to a patient in need of such treatment a compound of claim 1 in a therapeutically effective amount to treat the cellular proliferative disease.

42. The method of claim 41 wherein the cellular proliferative disease is cancer, hyperplasia, restenosis, cardiac hypertrophy, an immune disorder, a fungal disorder, or inflammation.

43. The method of claim 42 wherein the cellular proliferative disease is cancer.

44. (canceled)
